(19) United States
(12) Patent Application Publication
(10) Pub. No.: US 2009/0299049 A1
(43) Pub. Date: Dec. 3, 2009

(54) Bicyclic Compounds and Their Use

(76) Inventors: Ian Fairlamb, Heslington (GB); Stewart James, Heslington (GB); George Cristoph Baumann, York (GB); Graeme Andrew Firth, West Yorkshire (GB)

Correspondence Address: VINSON & ELKINS, L.L.P., FIRST CITY TOWER, 1001 FANNIN STREET, SUITE 2500, HOUSTON, TX, 77002-6760, US

(21) Appl. No.: 12/223,646

(22) PCT Filed: Feb. 7, 2007
(86) PCT No.: PCT/GB2007/000418

§ 371 Date: Jan. 12, 2009

(30) Foreign Application Priority Data
Feb. 7, 2006 (GB) 0602412.9

(51) Int. Cl. C07H 19/16

(52) U.S. Cl. 536/27.81

(57) ABSTRACT

The present invention provides fluorescent bicyclic compounds of the formula (I); wherein ring A, the broken lines -----, C₁----C₂, R⁴ and R¹ are as defined herein. The invention also relates to nucleoside and nucleotide analogues of said compounds, and their use as biological markers.

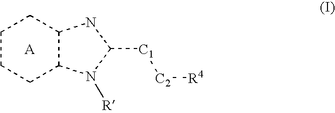

### FIELD OF THE INVENTION

The present invention relates to bicyclic ring systems, methods for making the same and uses thereof. In particular, the present invention relates to nucleoside and nucleotide analogues.

### BACKGROUND TO THE INVENTION

Nucleoside and nucleotide analogues are used in many applications, such as pharmaceutical agents and molecular probes. In particular, modified fluorescent nucleosides are valuable DNA and RNA structural probes, facilitating a greater understanding of protein recognition, biochemical pathways and the identification of new cellular processes.

Nucleoside 5′-phosphates (NTP) are necessary for many cellular processes, e.g. DNA, RNA and protein synthesis, energy metabolism, assembly of filamentous protein structures, and signal transduction. In their role as second messengers, during signal transduction, the purine NTPs are found in both cyclic nucleoside monophosphate and cyclic nucleoside diphosphate-ribose forms. In each cellular process, the base, ribose ring and phosphate group(s) have process-specific roles. As a consequence, these groups can be chemically altered to generate tailored fluorescent or photo-reactive NTP analogues that can be used to probe biochemical function in a process-specific manner.

Phosphate-, ribose-, and base-modified analogues of guanosine, and its cyclic derivatives, have been developed for use as fluorescent and photo-reactive probes. Coumarin and BODIPY probes have been attached via phosphoramidate and phosphorothioate linkages to the gamma-phosphate of GTP. Modifications to the ribose or modified ribose ring, e.g. 2′(3)-O-esters or 2′(3′)-O-carbamates, have also been used to attach probes. Fluorescent modifications of the ribosyl ring include 2′(3′)-O-(2,4,6-trinitrocyclohexadienylidene)- (TNP), 2′(3′)-O-(N-methyl)anthraniloyl- (MANT), and 2′(3′)-O-anthraniloyl- (ANT) groups. A plethora of fluorescent probes have been attached using a 2′(3′)-O-(2-aminoethylcarbamoyl)- (EDA) modified ribose ring. The spectral properties of the fluorophore are usually not altered by the chemical coupling, thus EDA-ribosyl modifications are frequently utilised. Modifications to the guanine ring at the 2-amino, 6-carbonyl, 7-imino and the 8-position result in derivatives with fluorescence or altered absorption characteristics. The potential for modification of the purine ring at the latter is vast, as a 8-halogenated derivative can be easily prepared and the halide displaced by an appropriate nucleophile, e.g. 8-(5-thioacetamidofluorescein)-cGMP.

Donor-acceptor phenylene-ethynylene substituted π-conjugated organics possessing low-lying charge-transfer excited states are known to exhibit a range of fluorescence properties. By lengthening, shortening, or broadening (through ring expansion) the π-conjugated electron network, it is possible to alter the properties of the fluorophore and tailor for a specific application. Conjugated organics of this type have been coupled to the 5-position of 2′-deoxyuridine, and to uridine-2′-carbamates.

Sessler et al (J. Am. Chem. Soc. 2001, 123, 3655-3660) disclose dimethylaniline-anthracene ensembles formed via Watson-Crick base pairing. Although the presence of the anthracene moiety imparts fluorescent properties to the compounds, the anthracene ring is considered too bulky for the compounds to be accepted into biological receptors. Furthermore, the utility of the compounds is limited by their emission wavelengths and solubility.

### SUMMARY OF THE INVENTION

The present invention is based at least in part on a discovery that the presence of a delocalised electron system in certain bicyclic compounds may provide unique and tunable fluorescence properties. The present compounds may be advantageous with respect to existing dyes because they may impart unique fluorescence properties to nucleosides without the addition of bulky moieties. The spectroscopic properties of the compounds may also be tailored by altering the electronic characteristics of particular substituents. Furthermore, due to the substituents employed, the present compounds may be less reactive under oxidising conditions than existing commercial dyes. Electrons that would normally be available for these reactions may be delocalised in the system. This may minimise side reactions in biological systems and the resulting changes in molecular structure that might otherwise lead to altered fluorescent properties.

In one aspect, the present invention provides compounds having a general formula (I):

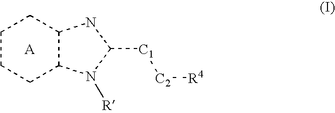

wherein
ring A is a six-membered carbocyclic or heterocyclic ring;
the broken lines ------ represent a delocalized electron system where electronic communication is possible;
C₁---C₂ is an alkenylene, alkynylene or thiophene linker;
R⁴ is a substituted or unsubstituted ring, e.g. selected from cyclopentadiene, phenyl, naphthyl, pyridine, pyrazine, pyrimidine, triazine, thiophene, parathiazine, pyrrole, pyrazole, imidazole, napthylene, indole, purine, benzimidazole, quinoline and phthalazine, any of which is substituted or unsubstituted; or R⁴ comprises a ring system comprising one or more metal atoms; and
R′ is a substituent selected from hydrogen, halogen, lower alkyl, halo-lower alkyl, carboxy, esterified carboxy, hydroxy, O-lower alkyl, etherified or esterified hydroxy, lower alkoxy, phenyl, substituted phenyl, lower alkanoyl, amino, mono- or di-substituted amino, amidino, ureido, mercapto, N-hydroxy-amidino, guanidino, amidino-lower alkyl, sulfo, SH, S-lower alkyl, sulfamoyl, carbamoyl, cyano, cyano-lower alkyl, azo (N═N═N), nitro and a sugar residue.

The present invention also provides a base-modified analogue of a nucleoside, its corresponding mono, di or triphosphorylated nucleotide, or its corresponding 3′,5′-cyclic monophosphate nucleotide derivative. Therefore, in a further aspect the present invention relates to compounds having the formula (II):

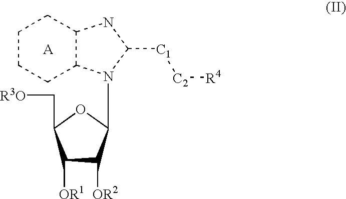

wherein R¹, R² and R³ are each independently selected from H, lower alkyl, halo-lower alkyl, carboxy, lower alkanoyl and a counter ion to the ion, O⁻ or one or more phosphate groups; or R¹ and R³ taken together may form a cyclic monophosphate derivative.

In a further aspect, the present invention provides compounds having a formula (IIIa):

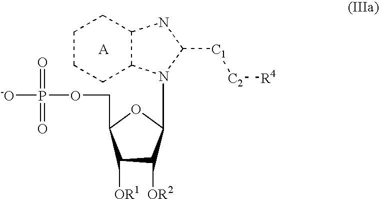

and further compounds having a 5′-diphosphate group (formula IIIb) or a 5′-triphosphate group (formula IIIc). The present invention also relates to processes for making compounds of formulae I to IIIa-c.

The compounds may be useful as fluorescent dyes. Thus, the present invention also relates to uses of the compounds of formulae I to IIIa-c as, for example, molecular fluorescent probes, substrates for DNA- and RNA-dependent DNA and RNA polymerases, substrates for nucleotidyl kinases, substrates for NTPases (where N=purine nucleotide), and as pharmaceutical agents against viral infections, pathogenic organisms and rapidly proliferating cell types. For example, the compounds may be used to treat abnormal conditions caused by hepatitis viruses, respiratory syncytial virus, Yellow fever virus, Dengue virus, West Nile virus, human cytomegalovirus, T-lymphotropic leukemia virus, herpes viruses, Epstein-Barr virus, papillomaviruses (HPV, HPV1 or HPV2), HIV virus, Plasmodium sp., Leishmania sp., Toxoplasma gondii, trypansomatid parasites, and rapidly proliferating ‘immortal’ cancer cells.

In particular, the present invention provides base-modified purine analogues, where the structure comprises C-modification at the 8-position.

The present invention also includes a combinatorial fluorophore array library which comprises multiple solid supports or multiple locations on a solid support, wherein each support or location has attached thereto an oligomer comprising the fluorescent nucleoside analog of a compound of the present invention.

In addition, the present invention also includes an oligonucleotide analog comprising one or more nucleoside analogs of a compound of the present invention in place of a DNA or RNA base.

The invention also provides a kit comprising, e.g. in separate containers, a compound of the invention and a solubility enhancing agent. The solubility enhancing agent may be a detergent. In particular, the solubility enhancing agent may be dimethylsulfoxide (DMSO) or dimethylformamide (DMF). Kits of the invention may be particularly useful in fluorescence assays.

### DESCRIPTION OF VARIOUS EMBODIMENTS

### Definitions

Halogen is especially fluorine, chlorine, bromine or iodine, more especially fluorine, chlorine or bromine, in particular fluorine.

Alkyl may have up to 20, for example up to 12, carbon atoms and may be linear or branched one or more times. Of mention is lower alkyl, especially preferred is C₁-C₄-alkyl, in particular methyl, ethyl or i-propyl or t-butyl, where alkyl may be substituted by one or more substituents.

Alkyl may be optionally interrupted by one or more in-chain heteroatoms, for example —O—, —S— or —N—, thus forming, for example, an ether, sulphide or amino/imino linkage.

Substituted alkyl is alkyl as last defined, especially lower alkyl, preferably methyl; where one or more, especially up to three, substituents may be present, primarily from the group selected from halogen, especially fluorine, amino, N-lower alkylamino, N,N-di-lower alkylamino, N-lower alkanoylamino, hydroxy, cyano, carboxy, lower alkoxycarbonyl, and phenyl-lower alkoxycarbonyl. Trifluoromethyl is especially preferred. One class of compounds includes a substituted alkyl where the alkyl is substituted with a heterocyclic ring, for example a pyrazine ring, thus forming an alkylene-het group, i.e. —CH₂-Het, the alkyl group effectively acting as a linker between the heterocycle and a second moiety.

The term “lower” when referring to substituents such as alkyl, alkoxy, alkyl amine, alkylthio and the like denotes a moiety having up to and including a maximum of 7, especially from 1 up to and including a maximum of 4, carbon atoms, the moiety in question being unbranched or branched one or more times.

The alkyl portion of lower alkyl, lower alkoxy, mono- or di-lower alkyl amino, lower alkyl thio and other substituents with an alkyl portion is especially C₁-C₄ alkyl, for example n-butyl, sec-butyl, tert-butyl, n-propyl, isopropyl, methyl or ethyl. Such alkyl substituents are unsubstituted or substituted by halogen, hydroxy, nitro, cyano, lower alkoxy, C₃, C₄, C₅, C₆ or C₇ cycloalkyl, amino, or mono- or di-lower alkyl amino, unless otherwise indicated.

Halo-lower alkyl, halo-lower alkyloxy, halo-lower alkylthio and the like refer to substituents having an alkyl portion wherein the alkyl portion is mono- to completely substituted by halogen. Halo-lower alkyl, halo-lower alkyloxy, halo-lower alkylthio and the like are included within substituted lower alkyl, substituted lower alkoxy, substituted lower alkylthio and the like.

Among the moieties corresponding to substituted alkyl, hydroxy-lower alkyl, especially 2-hydroxyethyl, and/or halo-lower alkyl, especially trifluoromethyl or 2,2,2-trifluoroethyl, or 1,1,2,2,2-pentaethyl are especially preferred.

As used herein, the term mercapto defines moieties of the general structure —S—Rₑ wherein Rₑ is selected from H, alkyl, a carbocylic group and a heterocyclic group as described herein.

As used herein, the term guanidino defines moieties of the general structure —NHR—C(NH)NH₂ and derivatives thereof; in particular, where hydrogen is replaced by alkyl, e.g. methyl or ethyl.

As used herein, the term amidino defines moieties of the general structure —C(NH)NH₂ and derivatives thereof, in particular, where hydrogen is replaced by alkyl, e.g. methyl or ethyl.

A mono- or di-substituted amino moiety may be defined where the amino is optionally substituted by a hydrocarbyl moiety, the hydrocarbyl moiety being, for example, selected from lower alkyl, especially C₁, C₂, C₃ or C₄ alkyl, cycloalkyl, especially cyclohexyl, alkyl-carboxy, carboxy, lower alkanoyl, especially acetyl, a carbocyclic group, for example cyclohexyl or phenyl, a heterocyclic group; where the hydrocarbyl moiety is unsubstituted or substituted by, for example lower alkyl (C₁, C₂, C₃, C₄, C₅, C₆ or C₇), halogen, OH, lower alkoxy, NH₂, SH, S-alkyl, SO-alkyl, SO₂-alkyl, NH-alkyl, N-dialkyl, carboxyl, CF₃, wherein alkyl may be optionally substituted branched, unbranched or cyclic C₁₋₆, interrupted 0-3 times by O, S, N.

Cycloalkyl may be C₃-C₁₀-cycloalkyl, especially cyclopropyl, dimethylcyclopropyl, cyclobutyl, cyclopentyl, cyclohexyl or cycloheptyl, cycloalkyl being unsubstituted or substituted by one or more, especially 1 to 3, substituents.

Esterified carboxy may be lower alkoxycarbonyl, such as tert-butoxycarbonyl, iso-propoxycarbonyl, methoxycarbonyl or ethoxycarbonyl, phenyl-lower alkoxycarbonyl, or phenyloxycarbonyl.

Alkanoyl may be alkylcarbonyl, especially lower alkanoyl, e.g. acetyl. In particular, the alkanoyl group may be substituted by substituents, e.g. CO—R

A sugar residue may be, for example, a sugar or derivatised sugar, such as an aminosugar, for example, and may be a pentose or a hexose, for example glucose, galactose, fructose or ribose. The sugar may be in hydroxylated or deoxy form. The sugar residue may be a monosaccharide and in certain compounds is a disaccharide. Other sugars are not excluded. Monosaccharide moieties may have 4, 5 or 6 carbon atoms, e.g. 6. In one preferred class of compounds, the sugar residue is an aldopentose or aldohexose. In another class of compounds, the sugar residue is a ketose, e.g. a ketohexose or ketopentose. The saccharides may be of D- or L-configuration. Exemplary sugars are ribose, arabinose, xylose, lyxose, allose, altrose, glucose, gulose, mannose, idose, galactose and talose. Also to be mentioned are mannose, galactose and fructose. The sugar residue may be in phosphorylated form, for example as described in formulae IIIa-c.

A cyclic group may be heterocyclic or carbocyclic.

A carbocyclic group may be a cycloalkyl, cycloalkenyl or aryl.

A heterocyclic group may be heterocyclyoalkyl, heterocycloalkenyl or aryl.

Cycloalkyl may be, for example, C₃-C₁₀-cycloalkyl, especially cyclopropyl, dimethylcyclopropyl, cyclobutyl, cyclopentyl, cyclohexyl or cycloheptyl, cycloalkyl being unsubstituted, or substituted by one or more, especially 1, 2 or 3, substituents.

Heterocyclyoalkyl is the same as cycloalkyl except that at least one of the in-ring carbon atoms is replaced with a heteroatom selected from N, O or S. The heteroatom may be N.

Cycloalkenyl and heterocycloalkenyl are the same as cycloalkyl and heterocyclyoalkyl respectively, except that they have at least one in-ring double bond, i.e. at least one degree of unsaturation.

Substituents of, for example, alkyl or cycloalkyl, may be selected form one or more, especially up to three, substituents primarily from the group selected from halogen, especially fluorine, amino, N-lower alkylamino, N,N-di-lower alkylamino, N-lower alkanoylamino, hydroxy, cyano, carboxy, lower alkoxycarbonyl, and phenyl-lower alkoxycarbonyl. Trifluoromethyl is especially preferred.

An aryl group is aromatic and may be heterocyclic aryl or carbocyclic aryl. In certain compounds, aryl has a ring system of not more than 16 carbon atoms and is preferably mono- bi- or tri-cyclic and may be fully or partially substituted.

A substituted carbocyclic aryl group is generally an aryl group that is substituted with from 1-5, e.g. 1 or 2, substituents. In certain compounds, a carbocyclic aryl group is selected from phenyl, naphthyl, indenyl, azulenyl and anthryl, and may in each case be unsubstituted or substituted by, for example, lower alkyl, especially methyl, ethyl or n-propyl, halo (especially fluoro, chloro, bromo or iodo), substituted lower alkyl, for example halo-lower alkyl (especially trifluoromethyl), hydroxy, lower alkoxy (especially methoxy), substituted lower alkoxy, for example halo-lower alkoxy, or amino-lower alkoxy, lower alkanoyl, carbamoyl, N-mono- or N,N-di-lower alkyl substituted carbamoyl, wherein the lower alkyl substituents may be unsubstituted or further substituted, for example lower alkyl (especially methyl or ethyl) carbamoyl, amino, mono- or di-lower alkyl substituted amino, wherein the lower alkyl substituents may be unsubstituted or further substituted by those substituents listed above for alkyl groups, nitro, cyano, mercapto, lower alkylthio, halo-lower alkylthio, heterocyclyl, heteroaryl, heterocyclylalkyl or heteroarylalkyl.

Heterocyclic moieties (aryl and non-aryl) may, for example, be selected from the group consisting of oxiranyl, azirinyl, 1,2-oxathiolanyl, imidazolyl, thienyl, furyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, pyranyol, thiazolyl, isothiazolyl, dithiazolyl, oxazolyl, isoxazolyl, pyridyl, pyrazinyl, pyrimidinyl, piperidyl, piperazinyl, pyridazinyl, morpholinyl, thiomorpholinyl, indolizinyl, isoindolyl, 3H-indolyl, indolyl, benzimidazolyl, cumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxalyl, quinazolinyl, quinazolinyl, cinnolinyl, pteridinyl, carbazolyl, β-carbolinyl, phenanthridinyl, acridinyl, perimidinyl, phenanthrolinyl, furazanyl, phenazinyl, phenothiazinyl, phenoxazinyl, chromenyl, isochromanyl and chromanyl, each of these radicals being unsubstituted or substituted by one to two substituents as defined herein.

The term “electronic communication” as used herein includes reference to the transfer or delocalisation of electrons in a x-system, for example, and may be exemplified by the presence of inductive and mesomeric effects within the compounds as described herein.

Any reference to compounds, salts and the like in the plural is always to be understood as including one compound, one salt or the like.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

### Compounds

Compounds of the present invention have a general formula (I):

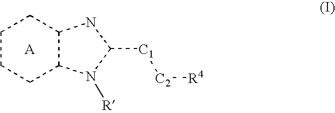

wherein
ring A is a six-membered carbocyclic or heterocyclic ring;
the broken lines ------ represent a delocalized electron system where electronic communication is possible;
C₁---C₂ is an alkenylene, alkynylene or thiophene linker,
R⁴ is a substituted or unsubstituted ring, e.g. selected from cyclopentadiene, phenyl, naphthyl, pyridine, pyrazine, pyrimidine, triazine, thiophene, parathiazine, pyrrole, pyrazole, imidazole, napthylene, indole, purine, benzimidazole, quinoline and phthalazine, any of which is substituted or unsubstituted; or e.g. comprises a ring system comprising one or metal atoms (e.g. a substituted or unsubstituted ferrocenyl group); and
R′ is a substituent selected from hydrogen, halogen, lower alkyl, halo-lower alkyl, carboxy, esterified carboxy, hydroxy, O-lower alkyl, etherified or esterified hydroxy, lower alkoxy, phenyl, substituted phenyl, lower alkanoyl, amino, mono- or di-substituted amino, amidino, ureido, mercapto, N-hydroxy-amidino, guanidino, amidino-lower alkyl, sulfo, SH, S-lower alkyl, sulfamoyl, carbamoyl, cyano, cyano-lower alkyl, azo (N═N═N), nitro and a sugar residue.

### Ring A

Ring A may, for example, be aromatic and may be substituted or unsubstituted.

In one class of compounds of the present invention, ring A is heterocyclic, for example containing at least one heteroatom selected from N, O and S.

In a further class of compounds, ring A comprises two nitrogen atoms.

Exemplary ring A structures are shown below, where ring A contains two nitrogen atoms and is aromatic:

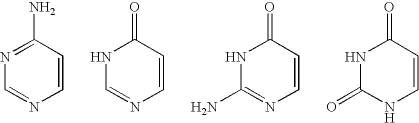

Ring A may be substituted to alter the electron donating or electron withdrawing properties of the ring, for example the purine ring as shown above.

Other examples of substituted rings are where any hydrogen bonded to either a nitrogen, oxygen or carbon atom is replaced by any of lower alkyl, lower acyl or a sugar residue, for example.

### Bicyclic Ring System

In certain compounds, the bicyclic system is a purine system. In other words, the bicyclic system includes any of the following structures A, B, C and D:

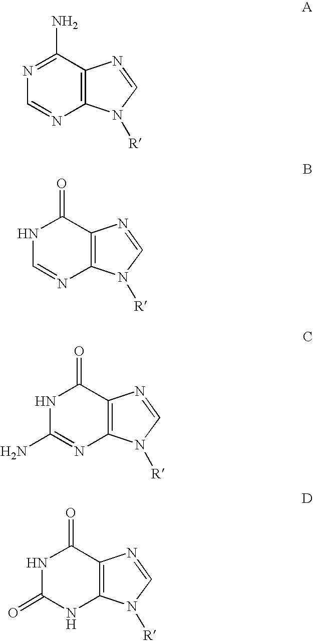

The bicyclic system may be substituted as described herein. In particular, and by way of example, replacement of any hydrogen atom may be made by a lower alkyl, lower acyl or a sugar residue.

### Electronic Communication

Compounds of the invention comprise an electron system whereby electronic communication is possible. The transfer of electrons may, for example, be delocalized through a conjugated x-network (alkenyl, alkynyl or aryl). In other words, the compounds of the present invention may express significant mesomeric and/or inductive characteristics. Electronic communication may in some cases be possible between Ring A and R⁴.

Mesomeric effects refer to the delocalisation of electrons from one atom or group of atoms into a π-conjugated system, e.g.

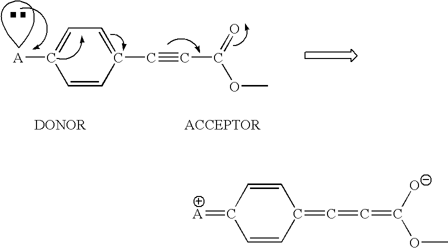

Inductive effects refer to the removal or donation of electron density by an atom or group of atoms in any bonded system (a through bond effect), which alters the partial charges (e.g. δ+ and δ−) in a given molecule, e.g.

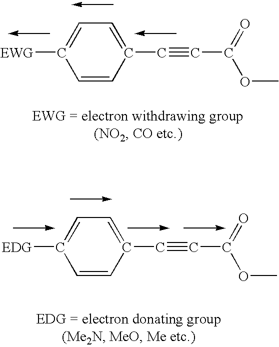

These characteristics may provide compounds of the present invention with useful properties, such as fluorescent properties, for example.

In addition, compounds having delocalized electron transfer may be less reactive than other known nucleoside and nucleotide analogues. This reduced reactivity therefore reduces the occurrence of side reaction and possible conformational or structural change of the analogue, which could lead to false or inaccurate readings when the compounds are being used as molecular probes.

Therefore, compounds of the present invention may be advantageous in that they are conformationally restricted. In one particular example, the compounds of the present invention exhibit rigid structures, where the delocalized network (-----) prevents substantial, preferably any, rotation about most, preferably all, bonds. Such compounds may be described as comprising an organic rigid-rod.

The delocalised network, where present, therefore provides the bonds with a partial double-bond character, similar to that associated with an amide bond.

In addition, the reduced reactivity makes the compounds of the present invention ideal candidates as enzyme substrates and pharmaceutical agents, for example, as molecular fluorescent probes, substrates for DNA- and RNA-dependent polymerases, substrates for nucleotidyl kinases and substrates for NTPases (where N=purine nucleotide).

The broken lines ------ may represent partial double bond character and/or partial charge character, which may also be represented by isomeric and/or mesomeric forms of a given compound and/or the presence of inductive effects, giving rise to partial charge characteristics, in the form of centres of positive (e.g. δ+) and negative (e.g. δ−) charge, as shown above.

In some classes of the compounds of the present invention, the delocalized system is present over the entire molecular structure. In other words, the entire molecule including the moiety C₁---C₂, the ring structure R⁴ and any substituents thereon forms a conjugated system where delocalized electrons pass throughout the whole molecule.

In another class of compounds, the delocalised system is present through the moiety C₁---C₂ and the ring structure R⁴ and may be influenced by any substituent associated therewith through any or both of mesomeric and inductive effects of that substituent.

Therefore, in one aspect of the present invention, it is possible to “tune” a compound by way of interchanging one or more substituents thereof to provide or introduce one or more mesomeric and/or inductive effects, which may in turn alter the properties of the compound. An example of such properties are the reactivity of a compound and/or its ability to fluoresce and/or its intensity of fluorescence. In some classes of compounds, these properties are all inter related.

The bicyclic structure comprising ring A is also part of the delocalised system in a particularly preferred set of compounds.

In one class of compounds all of the in-ring bonds of the bicyclic structure, the moiety C₁---C₂ and the in-ring bonds of the ring structure R⁴ have partial double bond characteristics.

### C₁----C₂

In certain compounds, the moiety C₁----C₂ is in conjugation with the ring bonds in the molecule, for example as represented by the broken lines -----. In particular, the moiety may be conjugated with the bicyclic system, for example the heterocyclic (guanine or adenosine) ring system and/or the group R⁴ and/or any substituents associated therewith.

The moiety C₁----C₂ is generally an alkenylene, alkynylene or thiophene linker. In one set of compounds the moiety C₁---C₂ is an alkynylene linker. The invention includes compounds in which the linker forms part of a metal complex, e.g. a trans-Pt(phosphine)₂ complex of the type C₁-Pt(PZ₃)₂-C₂ (where each Z is independently an aromatic, cycloalkenyl, cycloalkanyl or alkyl group). Alternatively, the C₁---C₂ linker may be spanned by a dicobalt(0)hexacarbonyl moiety.

### R⁴

In certain compounds, R⁴ is a moiety which alters the electron flow via inductive or mesomeric effects. In one class of compounds, the mesomeric effects dominate. In another class of compounds, exploitable inductive effects may dominate, for example in the case of trifluoromethyl, which may be due to the strongly electron-withdrawing nature of this substituent.

In one embodiment, R⁴ is a substituted or unsubstituted unsaturated cyclic group, for example selected from cyclopentadiene, phenyl, naphthyl, pyridine, pyrazine, pyrimidine, triazine, thiophene, parathiazine, pyrrole, pyrazole or imidazole, napthaline, indole, purine, benzimidazole, quinoline or phthalazine.

Each ring system contains π-electrons that can be delocalised (e.g. which can be made to transfer from the highest occupied molecular orbital (HOMO) to the lowest unoccupied molecular orbital (LUMO); the energy difference between these orbitals will change depending on the type of R-group and structure of the unsaturated cyclic linker group).

In embodiments, R⁴ is selected from one or more of phenyl, cyclopentadiene, napthyl and thiophene.

In other embodiments, R⁴ is a substituted or unsubstituted five or six membered ring.

In a particular class of compounds, the ring structure R⁴ interacts with the delocalized system (----). In this class, R⁴ may be aryl, for example.

The R⁴ group, e.g. phenyl, may be substituted by one or more substituents T. The or each T may, for example, be independently selected from halogen; hydroxy; protected hydroxy for example trialkylsilylhydroxy or etherified or esterified hydroxy; amino; mono- or di-substituted amino; amidino; guanidino; hydroxyguanidino; formamidino; N-hydroxy amidino; isothioureido; ureido; mercapto; C(O)H or other lower acyl; lower acyloxy; carboxy; esterified carboxy; sulfo; sulfamoyl; carbamoyl; cyano; azo (N═N═N); nitro; O-lower alkyl; or lower alkyl; O-lower alkyl and lower alkyl optionally substituted by one or more halogens and/or one or two functional groups selected from hydroxy, protected hydroxy for example trialkylsilylhydroxy, amino, mono- or di-substituted amino, amidino, guanidino, hydroxyguanidino, formamidino, isothioureido, ureido, mercapto, C(O)H or other lower acyl, lower acyloxy, carboxy, sulfo, sulfamoyl, carbamoyl, cyano, azo, or nitro; all of which hydroxy, amino, amidino, guanidino, hydroxyguanidino, formamidino, isothioureido, ureido, mercapto, carboxy, sulfo, sulfamoyl, carbamoyl and cyano groups being optionally substituted, where appropriate. Each T substituent may also be substituted, where appropriate. For example, amino lower alkyl may be substituted by hydroxyl or a halogen. Exemplary substituted alkyl groups include trifluoromethyl and polyfluorinated alkyl chains.

Of mention are substituents to the R⁴ cyclic group which interact with the delocalized electron system, e.g. the π-system, as depicted by the broken lines ----- in formula (I).

In one class of compounds the R⁴ group provides one or both of an inductive or mesomeric effect on the π-system of the bicyclic system, through the bond C₁---C₂. Therefore it may be preferable in these cases that the bond C₁---C₂ is in conjugation (i.e. has electron overlap) with both the R⁴ group and the bicyclic system. It may be preferable that, where substituted by one or more T groups, at least one T group interacts with the delocalized system (------).

The C₁---C₂ bond may be spanned or connected to a metal-containing species to alter to the electronic properties of the compound. In one class of compounds, R⁴ comprises a ring system comprising one or more metal atoms, for example one or more metal centres. Said ring system may be substituted or unsubstituted. In a particular class of compounds, R⁴ comprises a ferrocenyl group.

The present invention also provides a base-modified analogue of a nucleoside, its corresponding mono, di or triphosphorylated nucleotide, or its corresponding 3′,5′-cyclic monophosphate nucleotide derivative. Therefore, in a further aspect the present invention relates to compounds, having the formula (II):

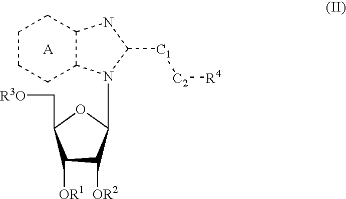

wherein R¹, R² and R³ are each independently selected from hydrogen, lower alkyl, halo-lower alkyl, carboxy, lower alkanoyl and a counter ion to the ion, O⁻ or one or more phosphate groups; or R¹ and R³ taken together may form a cyclic monophosphate derivative.

In a further aspect, the present invention relates to compounds having a formula (IIIa):

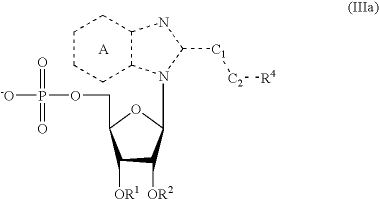

wherein R¹ and R² are each independently selected from hydrogen, lower alkyl, lower alkanoyl and a counter ion to the hydroxide anion, O⁻.

The invention also relates to further compounds having a 5′-diphosphate group (formula IIIb) or a 5′-triphosphate group (formula IIIc). The present invention also relates to processes for making compounds of formulae I to IIIa-c.

In one aspect of the present invention, compounds having a T group in place of the R⁴ group as described herein may be used as intermediates in the synthesis of the compounds of the present invention. In such a synthesis a T group may be displaced with an R⁴ moiety to provide a compound of the present invention, as described herein. An exemplary class of intermediates comprise of a halogen, where the process may comprise the step of displacing the halogen with an R⁴ moiety to form a compound of the present invention, by way of processes known in the art.

A particular class of compounds of the present invention has the following formula (IV):

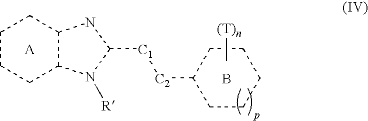

wherein
Ring B is a five- or six-membered carbocyclic or heterocyclic ring,
the or each T is independently selected from halogen; hydroxy; protected hydroxy for example trialkylsilylhydroxy or etherified or esterified hydroxyl; amino; mono- or di-substituted amino; amidino; guanidino; hydroxyguanidino; formamidino; N-hydroxy amidino; isothioureido; ureido; mercapto; C(O)H or other lower acyl; lower acyloxy; carboxy; esterified carboxy; sulfo; sulfamoyl; carbamoyl; cyano; azo; or nitro; O-lower alkyls and lower alkyl optionally substituted by one or more halogens and/or one or two functional groups selected from hydroxy, protected hydroxy for example trialkylsilylhydroxy, amino, mono- or di-substituted amino, amidino, guanidino, hydroxyguanidino, formamidino, isothioureido, ureido, mercapto, C(O)H or other lower acyl, lower acyloxy, carboxy, sulfo, sulfamoyl, carbamoyl, cyano, azo, or nitro; all of which hydroxy, amino, amidino, guanidino, hydroxyguanidino, formamidino, isothioureido, ureido, mercapto, carboxy, sulfo, sulfamoyl, carbamoyl and cyano groups being optionally substituted;
n is 1, 2, 3, 4, 5 or 6 or up to the maximum number of valent positions on the ring B; and
p is 0 or 1.

Ring B may interact with the delocalized system of the bicyclic ring and C₁----C₂. An example of such interaction is the delocalization of electrons between ring B and C₁----C₂, such as mesomeric interaction.

The substituent T may influence the absorbance properties of the compound.

In one embodiment of the present invention, there is provided compounds of formula (V):

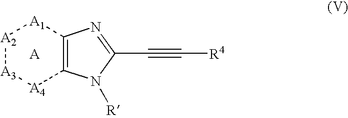

wherein R⁴ is as herein defined and A₁, A₂, A₃ and A₄ are each independently selected from C and N and may each independently be substituted by a substituent independently selected from H, NR^{a}R^{b}, ═O, alkyl, acyl and a sugar moiety; where R^{a} and R^{b} are each independently selected from H, alkyl, acyl, NH₂ and a sugar moiety.

In certain compounds, A₂ and A₄ are N and A₁ and A₃ are C. A₁ and A₃ may also be independently selected from CH₂, C═O, C═S, or C═N—R^{a}, where R^{a} is selected from H, alkyl, acyl, NH2 and a sugar moiety. A₂ and A₄ may also be independently selected from CH₂, NR^{a}, PH, S, or O, where R^{a} is selected from H, alkyl, acyl, NH₂ and a sugar moiety.

The ring A may be substituted to alter the electron donating or electron withdrawing properties of the ring, for example the purine ring, specifically, at positions A₁, A₂, A₃, or A₄.

In a further embodiment, the present invention provides compounds of formulae (VIa-d), which are purines:

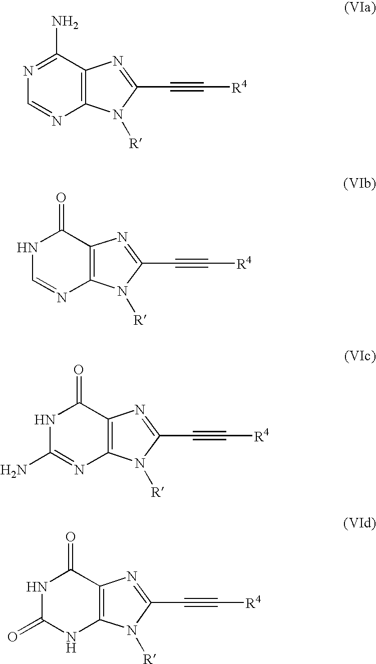

In embodiments, R′ is selected from hydrogen, lower alkyl, lower acyl and a sugar residue, for example.

In a further embodiment, a class of compounds of the present invention have the formula (VIIa-d):

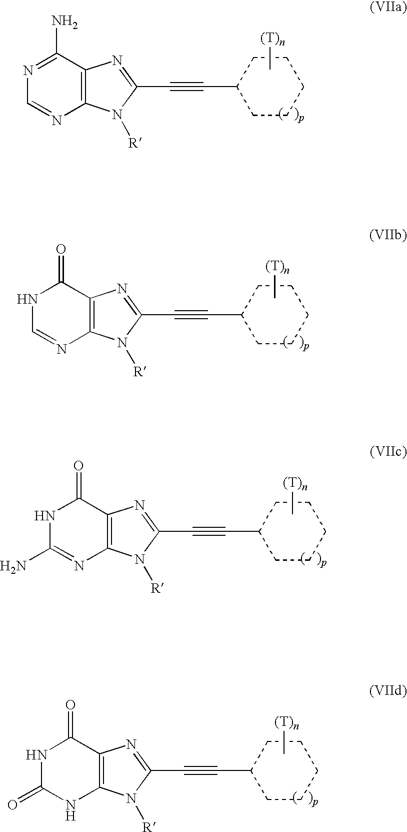

One particular class of compounds comprises ribose as a sugar residue. In another class of compounds, R′ is ribose or deoxy ribose.

Of particular mention are compounds having formulae VIIa or VIIc.

In another embodiment of the present invention, the R⁴ group is a substituted or unsubstituted phenyl, thus having the formula (VIIIa-d):

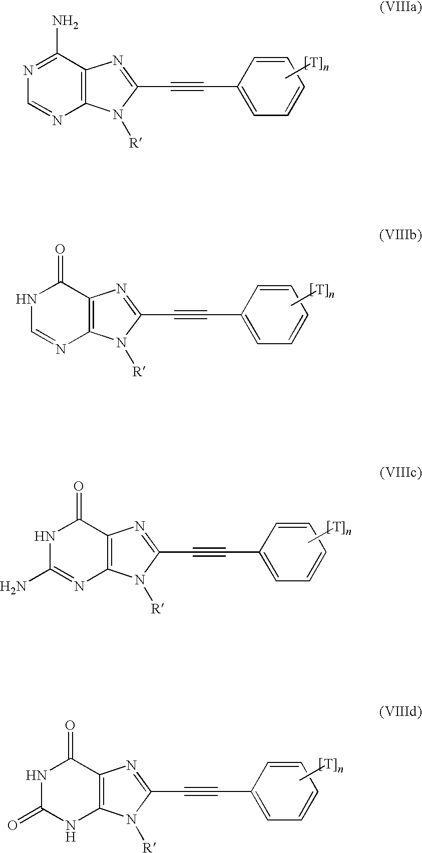

wherein
the or each T is independently selected from halogen; hydroxy; protected hydroxy for example trialkylsilylhydroxy or etherified or esterified hydroxyl; amino; mono- or di-substituted amino; amidino; guanidino; hydroxyguanidino; formamidino; N-hydroxy amidino; isothioureido; ureido; mercapto; C(O)H or other lower acyl; lower acyloxy; carboxy; esterified carboxy; sulfo; sulfamoyl; carbamoyl; cyano; azo; or nitro; O-lower alkyls and lower alkyl optionally substituted by one or more halogens and/or one or two functional groups selected from hydroxy, protected hydroxy for example trialkylsilylhydroxy, amino, mono- or di-substituted amino, amidino, guanidino, hydroxyguanidino, formamidino, isothioureido, ureido, mercapto, C(O)H or other lower acyl, lower acyloxy, carboxy, sulfo, sulfamoyl, carbamoyl, cyano, azo, or nitro; all of which hydroxy, amino, amidino, guanidino, hydroxyguanidino, formamidino, isothioureido, ureido, mercapto, carboxy, sulfo, sulfamoyl, carbamoyl and cyano groups being optionally substituted; and
n is 0, 1, 2, 3, 4 or 5.

In certain compounds, n is 1.

In a particular group of compounds, n is 1 and T is in the ortho or para position (particularly for electron-donating substituents) and meta position (particularly for electron-withdrawing substituents).

Of mention are compounds in which T (an electron-donating group or electron-withdrawing group, for example) is in the para position. Therefore, a particularly preferred class of compounds of the present invention have the formulae IXa-d:

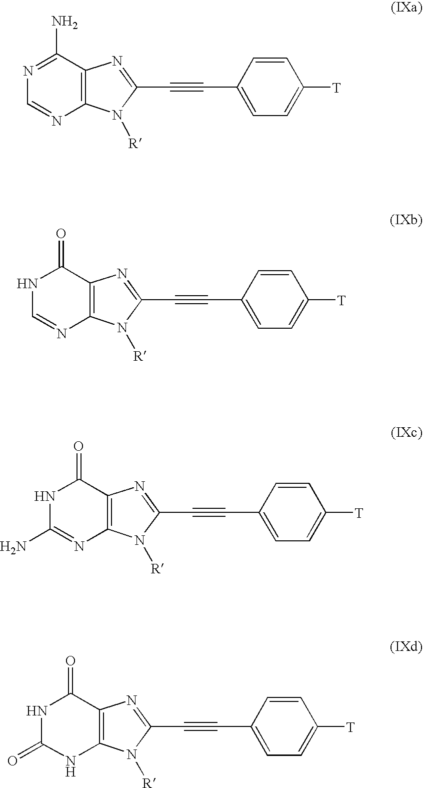

By way of example, the or each T may be independently selected from halogen; hydroxy; protected hydroxy for example trialkylsilylhydroxy or etherified or esterified hydroxyl; amino; mono- or di-substituted amino; amidino; guanidino; hydroxyguanidino; formamidino; N-hydroxy amidino; isothioureido; ureido; mercapto; C(O)H or other lower acyl; lower acyloxy; carboxy; esterified carboxy; sulfo; sulfamoyl; carbamoyl; cyano; azo (N═N═N); or nitro; O-lower alkyls and lower alkyl optionally substituted by one or more halogens and/or one or two functional groups selected from hydroxy, protected hydroxy for example trialkylsilylhydroxy, amino, mono- or di-substituted amino, amidino, guanidino, hydroxyguanidino, formamidino, isothioureido, ureido, mercapto, C(O)H or other lower acyl, lower acyloxy, carboxy, sulfo, sulfamoyl, carbamoyl, cyano, azo, or nitro; all of which hydroxy, amino, amidino, guanidino, hydroxyguanidino, formamidino, isothioureido, ureido, mercapto, carboxy, sulfo, sulfamoyl, carbamoyl and cyano groups being optionally substituted, where appropriate.

Exemplary T groups are NR^{a}R^{b} (wherein R^{a} and R^{b} are each independently selected from H, C₁, C₂, C₃, C₄ or C₅ alkyl, OMe, OEt, SMe, OH, SH, C₁, C₂, C₃, C₄ or C₅ alkyl, H, F, Cl, Br, I, CF₃, NO₂, COR (R=C₁, C₂, C₃, C₄ or C₅ alkyl, CO₂R (R=C₁, C₂, C₃, C₄ or C₅ alkyl), CN. In one particular class of compounds, T is selected from NR^{a}R^{b}, NEt₂ and H.

In a further embodiment of the present invention, T may be another cyclic group as herein defined. For example, T may be phenyl. In another embodiment, the C₁----C₂—R⁴ moiety may be a repeating unit. Therefore, the present invention also includes compounds of the formula (X)

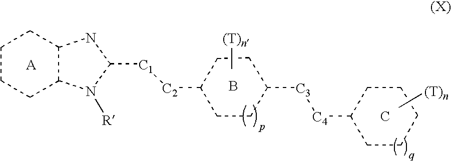

wherein
rings B and C are each independently five- or six-membered carbocyclic or heterocyclic rings;
C₃----C₄ moiety may, for example, be conjugated between the ring systems;
p and q are independently selected from 0 and 1;
n is 0, 1, 2, 3, 4 or 5; and
n′ is 0, 1, 2, 3 or 4

In certain compounds, C₃----C₄ is an alkenylene, alkynylene or thiophene linker.

In one class of compounds, the moieties C₁----C₂ and C₃----C₄ are the same, for example an ethyne linker group.

This may also be represented by formula (XI)

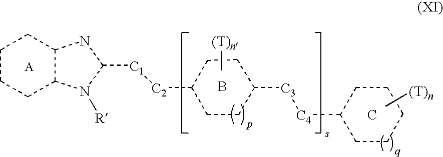

where s may be 0 or 1 and the other variables are as hereinbefore defined.

In compounds of the present invention the or each T may be the same or different. In other words, it will be understood that the T groups of any of rings B or C may be the same or different, i.e. independently selected.

In a particular class of compounds of the present invention, both the C₁----C₂ and the C₃----C₄ are an alkynyl linker:

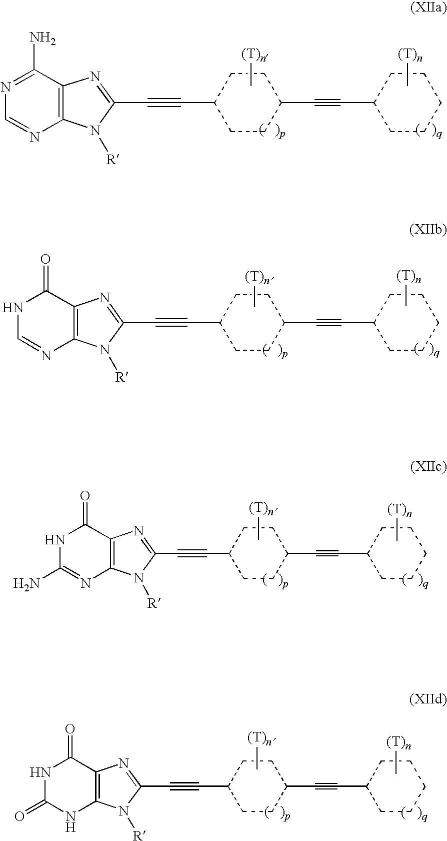

wherein the variable moieties are as hereinbefore defined.

As an example, taking the repeating unit [RING-C₁-----C₂-RING] as defined in formula (XII) and applying it to a more specific definition of the R⁴ group, a group of compounds having a formulae (XIIIa-d) may be generated.

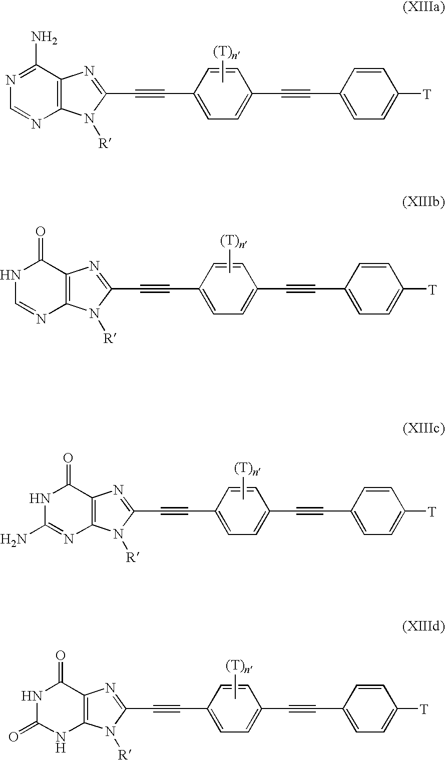

where T, n and R′ are as hereinbefore defined.

Some compounds of the formula may exist in the form of solvates, for example hydrates, which also fall within the scope of the present invention.

Compounds of the invention may be in the form of pharmaceutically acceptable salts, for example, addition salts of inorganic or organic acids. Such inorganic acid addition salts include, for example, salts of hydrobromic acid, hydrochloric acid, nitric acid, phosphoric acid and sulphuric acid. Organic acid addition salts include, for example, salts of acetic acid, benzenesulphonic acid, benzoic acid, camphorsulphonic acid, citric acid, 2-(4-chlorophenoxy)-2-methylpropionic acid, 1,2-ethanedisulphonic acid, ethanesulphonic acid, ethylenediaminetetraacetic acid (EDTA), fumaric acid, glucoheptonic acid, gluconic acid, glutamic acid, N-glycolylarsanilic acid, 4-hexylresorcinol, hippuric acid, 2-(4-hydroxybenzoyl)benzoic acid, 1-hydroxy-2-naphthoic acid, 3-hydroxy-2-naphthoic acid, 2-hydroxyethanesulphonic acid, lactobionic acid, n-dodecyl sulphuric acid, maleic acid, malic acid, mandelic acid, methanesulphonic acid, methyl sulphuric acid, mucic acid, 2-naphthalenesulphonic acid, pamoic acid, pantothenic acid, phosphanilic acid ((4-aminophenyl)phosphonic acid), picric acid, salicylic acid, stearic acid, succinic acid, tannic acid, tartaric acid, terephthalic acid, p-toluenesulphonic acid, 10-undecenoic acid and the like.

Salts may also be formed with inorganic bases. Such inorganic base salts include, for example, salts of aluminium, bismuth, calcium, lithium, magnesium, potassium, sodium, zinc and the like. Organic base salts include, for example, salts of N,N′-dibenzylethylenediamine, choline (as a counterion), diethanolamine, ethanolamine, ethylenediamine, N,N′-bis(dehydroabietyl)ethylenediamine, N-methylglucamine, procaine, tris(hydroxymethyl)aminomethane (“TRIS”) and the like.

It will be appreciated that such salts, provided that they are pharmaceutically acceptable, may be used in therapy. Such salts may be prepared by reacting the compound with a suitable acid or base in a conventional manner.

The compounds of the invention can exist in different forms, such as free acids, free bases, esters and other prodrugs, salts and tautomers, for example, and the invention includes all variant forms of the compounds.

The extent of protection includes counterfeit or fraudulent products which contain or purport to contain a compound of the invention irrespective of whether they do in fact contain such a compound and irrespective of whether any such compound is contained in a therapeutically effective amount. Included in the scope of protection therefore are packages which include a description or instructions which indicate that the package contains a species or pharmaceutical formulation of the invention and a product which is or comprises, or purports to be or comprise, such a formulation or species.

### Synthesis

A compound of the invention may be prepared by any suitable method known in the art and/or by the following processes, by way of example, an overall reaction scheme is provided below:

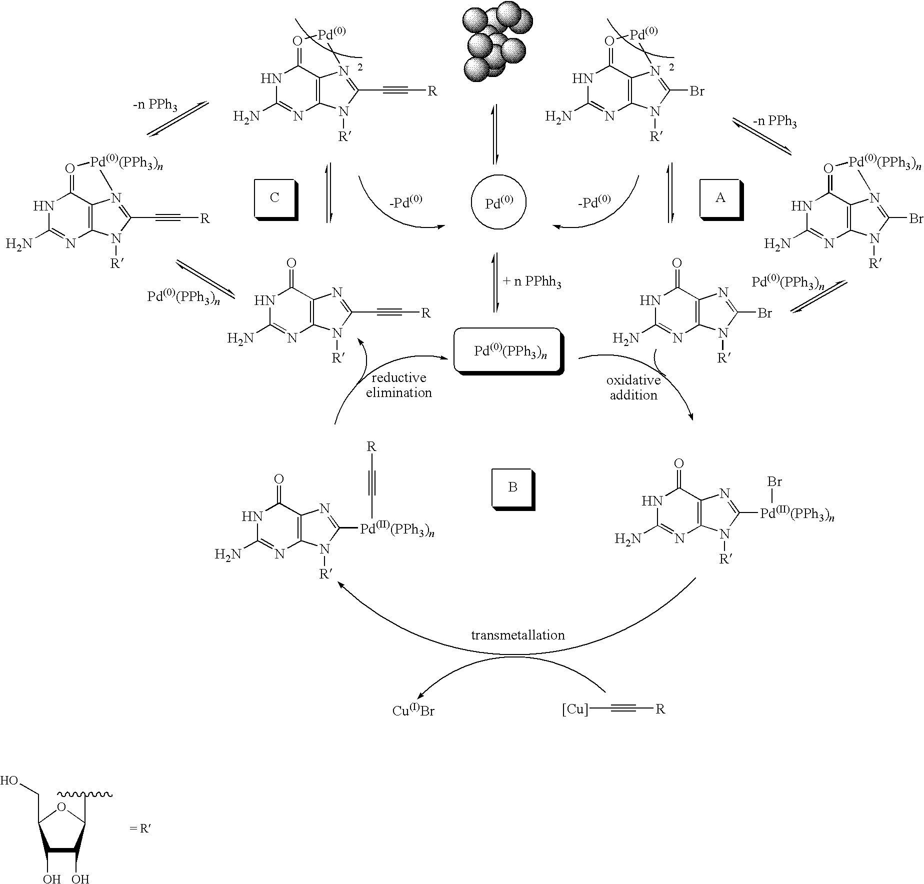

The above scheme takes into consideration the major catalytic cycle responsible for the formation of 8-alkynylatedguanosines using Sonogashira cross-coupling (Cycle B). Cycles A and B refer to competing catalytic cycles which removes catalytically active palladium from the reaction (which is in equilibrium with the reaction system). These cycles can be suppressed by increasing the copper(I) concentration (which competes with palladium coordination). At a certain concentration of palladium(0), agglomeration of the palladium is observed. Thus, the overall concentration of palladium is crucial to the extent and rate of the Sonogashira reaction.

The terminal acetylenes may be prepared using standard literature procedures or are commercially available for leading suppliers.

The following reaction scheme illustrates the key reaction that will be used for the formation of the modified purine base adduct, within the scope of the present invention:

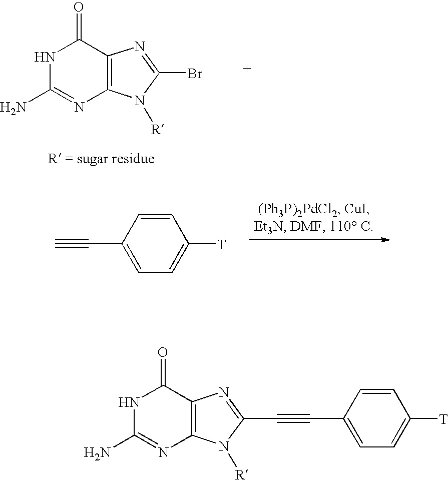

Alkynylated nucleosides can be accessed by Sonogashira cross-coupling, for example by alkynylation of the unprotected 8-bromoguanosine 1 with terminal acetylenes to give 5 in the presence of triethylamine (which functions as a base) and palladium(0) catalyst (generated in situ by pre-reduction of bis(triphenylphosphine)palladium(II) chloride) and a copper(I) co-catalyst (copper(I) iodide) in N,N-dimethylformamide at 110° C.

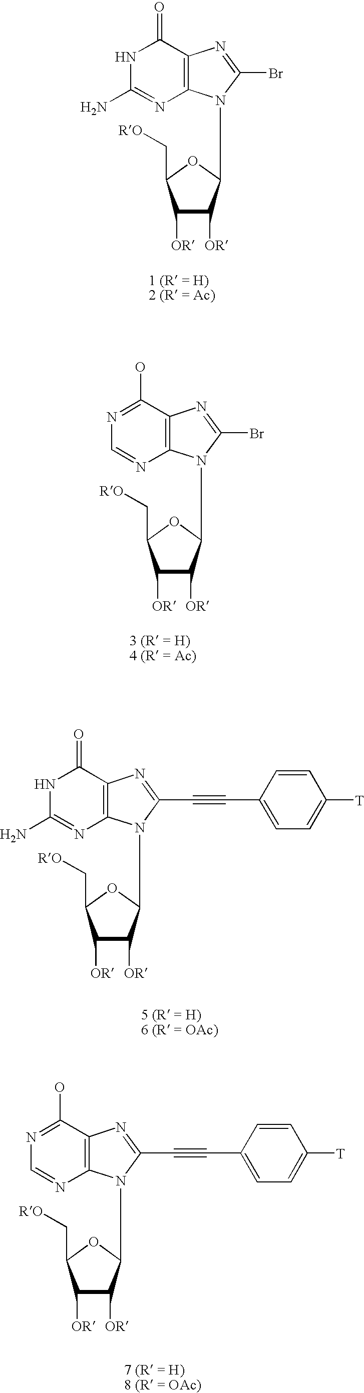

The Sonogashira alkynylation of 8-bromoadenosine to give two 8-alkynylated-adenosines may also be conducted.

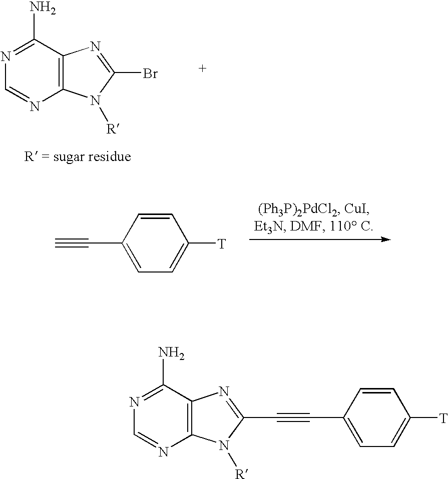

The hydroxyl substituents in guanosines (and adenosines) may be required for efficient cross-coupling. The substituents can be protected as the acetate derivatives, which improves solubility in most common, organic solvents (ethyl acetate, acetonitrile, dichloromethane, N,N-dimethylformamide etc.). However; it is noted that the process of the present invention does not require the sugar hydroxyl substituents to be protected. In fact, it is thought that protection may hinder the purification of these products since the reduced solubility may be enhanced to increase the purification process, for example by way of precipitation.

The Pd-mediated processes appear to be more facile with adenosine derivatives. The reason for this derives from the inherent higher activation of the adenosine heterocyclic ring system versus the more electron-rich guanine ring (which is relatively deactivated). Furthermore, various guanine coordination modes are possible, e.g. N-1/N-7 and O-6 coordination, to Pd (and presumably Cu), which could be a hindrance in the cross-coupling brominated guanosine derivatives.

It has been found that the copper(I) co-catalyst plays two roles in the reaction. The first is to assist in deprotonation of the terminal acetylene in the presence of triethylamine to generate the alkynyl cuprate (a reactive intermediate in the reaction which assists in transferring the alkynyl group onto the purine base adduct). A novel secondary role has been found for copper(I) which is related to the coordination of the guanine moiety to the palladium catalyst. In the absence of copper(I) it has been found that negligible reaction is observed. Whereas, in the presence of copper(I), in a ratio of 2:1 with palladium(0), results in efficient (optimal) reaction. The copper(I) competes with coordination to the guanine moiety which release active palladium(0) catalyst into the catalytic cycle, resulting in an increased rate of reaction.

It has been determined that the optimal concentration of the brominated purine bases is 0.1 to 0.14 M, typically 0.12 M (global concentration). The optimal ratio of terminal acetylene to brominated purine base is 1.2:1.

The optimal palladium concentration (global) has been determined to be about 1 mol %; c˜1×10⁻³ to 10⁻⁴ moldm⁻³. This relates to the formation of palladium colloids/clusters that are observed at higher palladium concentrations (confirmed by a mercury-drop test in Sonogashira reactions; shown by lower catalytic activity (turnover numbers and frequencies) at higher catalyst concentrations—an inverse correlation).

It will be understood that the processes detailed above are solely for the purpose of illustrating the invention and should not be construed as limiting. A process utilising similar or analogous reagents and/or conditions known to one skilled in the art may also be used to obtain a compound of the invention.

Any mixtures of final products or intermediates obtained can be separated on the basis of the physico-chemical differences of the constituents, in a known manner, into the pure final products or intermediates, for example by chromatography, distillation, fractional crystallisation, or by the formation of a salt if appropriate or possible under the circumstances.

### Use

Compounds of the invention may have utility in various applications, including, without limitation, the following applications.

### As Fluorescent Dyes

In an embodiment of the present invention the compounds provide a conjugated electronic system, which may provide unique and tunable fluorescence properties. The incorporation of a compound (e.g. a conjugated rigid-rod) of the present invention, attached directly by C-modification at the 8-position on the purine (guanosine and adenosine), represents a useful way to introduce a compact fluorescent moiety. These conjugated systems provide a distinct advantage over existing dyes because they may impart unique fluorescence properties to the purine nucleoside without the addition of bulky moieties.

A subset of compounds in the present invention contains phenylene-ethynylene linkages. These linear π-conjugated 8-alkynylated purine nucleosides may be designed to allow through-bond energy transfer. The spectroscopic properties of the present 8-alkynylated adenosines and guanosines show that the S₀ absorption wavelength and Stokes shift can be tuned by altering the electron-donating characteristics of the para-substituent on the phenyl ring. The large Stokes shifts observed are consistent with energy transfer to the guanine and adenine rings being enhanced by both electron-donating and electron-withdrawing substituents through the aromatic ring, respectively.

Due to the nature of the phenylene-ethynylene linkage, compounds of the present invention may be less reactive under oxidising conditions than existing commercial dyes. Electrons that would normally be available for these reactions are delocalised in the linkage. Therefore, this may minimise side reactions, and the resulting changes in molecular structure, that might lead to altered fluorescent properties.

The conjugated system provided by the compounds of the present invention may provide distinct advantages over existing dyes. Due to electronic communication derived from the double-bond character of the phenylene-ethynylene linkage, the compounds may be very rigid, e.g. an organic rigid rod (Chem. Comm. 2005, 2666-2668). The rigidity of the phenylene-ethynylene-purine system gives the fluorescent group at the 8-position on the purine base a fixed dipole relative to that of the purine ring. If the compounds are bound tightly by the active site of an enzyme, or a high-affinity site on an enzyme, or are incorporated into another rigid molecular structure, they may be used to probe the rotational motion of this complex, or the flexibility of the local environment, using fluorescence anisotropy spectroscopy. The analogues may be used as both in vitro and in vivo probes of this type.

The purine compounds of the present invention may be useful in studies of enzymes involved in the synthesis, modification, binding, polymerisation or hydrolysis of purine ribonucleotide triphosphate (NTP). Where the present fluorescent compounds are utilised as substrates by these enzymes (NTP converted to NDP+Pᵢ), they may be used as probes of NTP binding, and of conformational changes in the enzyme. NTP binding may be observed as a fluorescence intensity, anisotropy or emission wavelength change, where the enzyme is monitored at the single-molecule or the ensemble level. Fluorescence measurements of this type could be made in vitro or in vivo. Conformational changes may be detected by using the present fluorescent compounds as donor or acceptor dye molecules in fluorescence resonance energy transfer (FRET) experiments. FRET experiments could be accomplished by visualising single fluorophore molecules, e.g. total internal reflection fluorescence (TIRF) microscopy, or monitoring the fluorescence change in an ensemble of fluorophores.

The adenine and guanine compounds of the present invention may be useful as specific fluorescent probes of base pairing or base unpairing after incorporation into single-stranded DNA (ssDNA) or single-stranded RNA (ssRNA) oligonucleotides. Previous fluorescent modifications to the pyrimidine base of a ribonucleotide (Angew. Chem. Int. Ed. (2002) 41:3648-3650; Angew. Chem. Int. Ed. (2001) 40:1104-1106) and a deoxyribonucleotide (Tetrahedron Lett. (2004) 45:3543-3546) have been developed for similar applications. In this work, the fluorescence intensity of the incorporated pyrimidine analogues was enhanced upon complete hybridisation to a complementary oligonucleotide DNA or RNA sequence, respectively. In the case of the pyrimidine deoxyribonucleotide analogue, the fluorescence intensity was also sensitive to single-base-mismatch at the site of incorporation. The present compounds may be tested as sensitive probes of oligonucleotide hybridisation and single-base-mismatch. The compounds may also be used to incorporate fluorescent groups into dsDNA sequences via ssDNA oligonucleotide primers in polymerase chain reaction (PCR) applications. These may be monitored by using fluorescence microscopy; for example, in situ hybridisation (FISH) microscopy techniques.

By lengthening, shortening, or broadening (for example through ring expansion) the π-conjugated electron network, a skilled person can alter the properties of the fluorophore, in essence allowing one to tailor-make analogues for specific applications. The low lying charge transfer energy states of alkynyl/aryl groups, for example, in such derivatives may also be tuned through alteration of the para-substituent, for example, to modulate the fluorescence properties, thus minimizing the degree of steric changes to the parent phenyl-ethynyl-purine compound. Additional chemical substitution(s) may be introduced at the ortho and/or meta positions on the phenyl ring.

The compounds of the present invention are potential substrates for DNA- and RNA-dependent polymerases. The ability to follow NTP polymerisation by RNA and DNA polymerases using fluorescence techniques requires NTP analogues with a fluorophore attached to either the gamma phosphate or the base. Attachment of a fluorophore at either of these positions may allow complementary base pairing with the template DNA and phosphodiester bond formation, both required for polymerisation by the polymerase. NTP analogues with the fluorescent group attached to the base or sugar rings have the added benefit that the newly synthesised polynucleotide chain is rendered fluorescent. The commercially available fluorophores usually incorporate large, bulky fluorescent groups, e.g. cyanine or Alexa™dyes, which can interfere with the NTP polymerisation reaction. Ribosyl-analogues with a fluorophore attached directly to the purine base are not commercially available. The modified purine bases of the present invention may have a relatively compact fluorophore, which should be incorporated by RNA or DNA polymerases. Analogues of this type may be ideal for in vitro assays of incorporation into a polyribonucleotide chain or a polydeoxyribonucleotide chain, respectively. Analogues of this type may also be used in in vivo assays of incorporation by RNA or DNA polymerases. If incorporated by polymerases, the resulting polynucleotide chains would be fluorescent and could be detected by fluorescence anisotropy spectroscopy, fluorescence microscopy or UV-illumination/visible illumination of product bands on electrophoresis gels. The presence of these analogues in the polynucleotide chains may also be detected, or selected for, by preparing monoclonal or polyclonal antibodies to the present compounds. Antibodies to the present compounds may be used in many applications, some examples include Northern blotting, Southern blotting, Western blotting, immunoprecipitation, immunohistochemistry, flow cytometry, and enzyme-linked immunosorbent assays (ELISA).

The development of fluorescence-based polymerisation assays also reduces the existing need for radiolabelled substrate molecules in common assays of polymerisation. In addition, the inherent high sensitivity of fluorescence-based methods should allow the design of high throughput, multiplexed (96-well or 384-well format) assays.

Compounds of the present invention may be useful in the following types of applications:
substrate incorporation by DNA-dependent RNA polymerases in continuous (real-time) or dis-continuous (end-point) assays of ribonucleotide incorporation after triphosphorylation of the present inventions at the 5′-position;
substrate incorporation by RNA-dependent RNA polymerases in continuous (real-time) or discontinuous (end-point) assays of ribonucleotide incorporation after triphosphorylation of the present inventions at the 5′-position; and
substrate incorporation by DNA polymerases (with or without a requirement for a template strand) in continuous (real-time) or discontinuous (end-point) assays of deoxyribonucleotide incorporation after triphosphorylation of the present invention at the 5′-position.

In addition to the above, the present invention also includes the following methods:
A method of preparing a fluorescently labeled nucleic acid molecule which comprises incorporating at least one nucleoside analog of a compound of the present invention into an RNA or DNA molecule under conditions sufficient to incorporate said nucleoside;
A method of detecting a target nucleic acid in a sample to be tested which comprises contacting the target nucleic acid with a nucleic acid probe comprising at least one nucleoside analog of a compound of the present invention for a time and under conditions sufficient to permit hybridization between said target and said probe and detecting said hybridization;
A method of selecting a fluorophore suitable for use in labeling a nucleic acid molecule which comprises: constructing a combinatorial fluorophore array library of compounds of the present invention and selecting a fluorophore emitting the most intense fluorescence or emitting a specific wavelength of light;
A method of identifying a fluorophore emitting large Stokes shifts which comprises constructing a combinatorial fluorophore array library of compounds of the present invention, exciting the library at short wavelength, and selecting a fluorophore that emits light at a much longer wavelength; and
A method of identifying a fluorophore involved in energy transfer which comprises constructing a combinatorial fluorophore array library of compounds of the present invention, hybridizing a nucleic acid comprising a donor or acceptor dye to a nucleic acid sequence in the combinatorial fluorophore array library and correlating any change in color exhibited by the hybridized molecules with energy transfer.

Modification of position 8 on the purine base with a bulky functional group can also stabilise the syn conformer of these purine analogues. Normally, rotation about the purine-ribose (glycosidic) bond would allow both the anti and syn conformers to exist in solution. It is known that modification of the 8 position favours the syn conformer and promotes the formation of a self-assembling planar G-quartet structure (Angew. Chem. Int. Ed. (2000) 39: 1300-1302). The compounds of the present invention may be used to develop self-assembling nano-structures with unique fluorescent properties by incorporating them into poly(G) chains with riboGₙ or deoxyGₙ sequences. RNA or DNA G-quadruplex aptamers with fluorescent properties may be used as prototypes for biosensing nanostructures (Angew. Chem. Int. Ed. (2004) 43:668-698; see section 5), some examples include ligands in affinity probe capillary electrophoresis, ligands in evanescent-wave-induced fluorescence spectroscopy, aptamer beacons adopting two or more conformations, aptamer beacons for real-time protein recognition applications, and structure-switching signalling aptamers.

### As Inhibitors of Pathogen Replication

Adenine and guanine compounds of the present invention may act as inhibitors of pathogen (e.g. viral) replication, including replication of positive strand viruses and retroviruses. The efficacy of non-phosphorylated compounds from the present invention may be tested in an assay. For example, antiviral activity could be tested. By way of illustration, efficacy may be tested in an existing hepatitis C virus (HCV) replicon-based screening assay (J. Gen. Virol. (2002) 83:383-394; J. Gen. Virol. (2004) 85:429-439). Replicons are subgenomic, self-replicating RNA molecules that contain all the necessary coding sequence for RNA replication, transcription and translation. However, they are not infectious by themselves. The lipophilic and compact nature of the present compounds in the non-phosphorylated state may facilitate their uptake by cells in culture. After cellular uptake, the present compounds can be phosphorylated by endogenous nucleotidyl phosphorylation machinery. The present invention relates to nucleoside and nucleotide analog compounds that are capable of (i) exhibiting differential reactivity towards the pathogenic (e.g. viral) replicative enzymes relative to the human replicative enzymes, and (ii) effecting selective termination of the RNA replication process initiated by pathogenic (e.g. viral) enzyme(s), thereby resulting in the inability of the pathogen to replicate in the infected host without terminating chain replication by human polymerases. The compounds of the present invention may not interfere with processes initiated by human RNA and DNA polymerases, thereby rendering them substantially non-toxic to normal or uninfected cells.

The present compounds may directly inhibit the replicative viral RNA polymerase. An exemplary mechanism may be based on either (i) termination of RNA chain synthesis by the viral RNA polymerase (chain termination mechanism), or (ii) high-affinity active site binding (active site blockage mechanism). The present compounds may be easily altered to increase the affinity of an inhibitor, or decrease the cross-reactivity of an inhibitor with poor specificity. By altering the R-groups in formulae VIa-d, or the number of repeating units in formula XIII, for example, a skilled person can tune the affinity and specificity of the present compounds as inhibitors of viral polymerases and/or reverse transcriptases, for example viral RNA polymerase.

The present compounds may inhibit the proliferation of certain cell types, e.g. cancerous cells. In this capacity, the compounds may inhibit an enzyme or biological process key to cell proliferation, without affecting normally proliferating cell types. These compounds may be altered easily to increase the affinity of an inhibitor, or decrease the cross-reactivity of an inhibitor with poor specificity. By altering the R-groups in formulae VIa-d, or the number of repeating units in formula XIII, for example, a skilled person can tune the affinity and specificity of the present compounds as inhibitors of cell proliferation.

### Other Uses

Adenine compounds of the present invention may be useful with purine nucleoside phosphorylase (PNPase). This class of enzymes transfers an inorganic phosphate to either a non-phosphorylated purine ribofuranoside or a non-phosphorylated purine deoxyribofuranoside. The present compounds may act either as substrates for the hexameric (prevalent in bacteria) or trimeric (prevalent in eukaryotes) PNPases, or as inhibitors for these enzymes. Therefore, the present compounds may be used as fluorescent substrates in inorganic phosphate assay kits. A similar kit is already available using a completely different purine analogue (2-amino-6-mercapto-7-methyl-purine, Enzchek®Phosphate Assay kit, Molecular Probes). This kit can be used to detect the free inorganic phosphate concentration in an end-point type assay, or the kinetics of inorganic phosphate release by an enzyme in a real-time assay. The present compounds may be useful as inhibitors of bacterial and parasitic targets, e.g. Plasmodium falciparum, which are purine auxotrophs.

Adenine compounds of the present invention may be useful with DNA and RNA helicases that unwind dsDNA and dsRNA, respectively. Where the present fluorescent compounds are utilised as substrates by this class of enzymes (NTP converted to NDP+Pᵢ), they may be used as probes of NTP binding, and of conformational changes in the helicase. NTP binding may be observed as a fluorescence intensity, anisotropy or emission wavelength change, where the helicase is monitored at the single-molecule or the ensemble level. Fluorescence measurements of this type could be made in vitro or in vivo. Conformational changes may be detected by using the present fluorescent compounds as donor or acceptor dye molecules in fluorescence resonance energy transfer (FRET) experiments. FRET experiments could be accomplished by visualising single fluorophore molecules, e.g. total internal reflection fluorescence (TIRF) microscopy, or monitoring the fluorescence change in an ensemble of fluorophores.

Adenine compounds of the present invention may function as the required nucleotide triphosphate co-factor in the polymerisation of globular-actin (G-actin) to filamentous-actin (F-actin).

Hydrolysation by G-actin during the polymerisation reaction of the compounds of the present invention would produce F-actin polymers with fluorescent properties when irradiated with the appropriate wavelength of light. The movement of the resulting fluorescent F-actin polymers by surface-immobilised myosin proteins could be more easily tracked in in vitro (sliding-filament type) motility assays (Proc. Natl. Acad. Sci. USA (1986) 83:6272-6276; Nature (1987) 328:536-539) using epi-fluorescence or TIRF microscopy. Normally in these assays, F-actin would be stained with an extrinsic fluorophore to make the filament fluorescent, e.g. rhodamine-phalloidin. The present compounds may therefore allow F-actin to be viewed directly by epi-fluorescence or TIRF microscopy without this staining step. In motility assays, the direct action of myosin protein ensembles can be followed in real-time by tracking the motion of the fluorescent F-actin along the surface of a microscope cover slide. The present purine compounds may also bind to actin-like or tubulin-like protein monomers from prokaryotic, archaeal or eukaryotic sources and alter the equilibrium concentrations of the globular and filamentous forms of the protein.

The present compounds may be modified to incorporate a chemical moiety to function as an ‘electrochemical switch’. For example, if R⁴ comprises a ferrocene moiety (e.g. in in formulae VIa-d), the compounds may be used as cyclic voltametric probes. Fe(II) can be easily oxidised to Fe(III) and reduced back to Fe(II). Where a ferrocene derivative of the present compound was immobilised on a surface by a receptor, an enzyme, or a cell, the conducting properties of the buffer solution would change in a manner that is proportional to the immobilised compound's concentration after washing away the unbound compound. In this application, the present compounds may be used as sensitive probes of association and dissociation in an assay.

The compounds of the present invention may be used as pharmaceutical agents. The compounds of the invention may be used in the treatment of numerous ailments, conditions and diseases.

The compounds of the present invention may be useful as antiproliferatives, i.e. in the treatment of a proliferative disorder, for example, anti-cancer agents and agents for the treatment of psoriasis. In this capacity, the compounds may inhibit an enzyme or biological process key to cell proliferation, without affecting normally proliferating cell types. These compounds are altered easily to increase the affinity of an inhibitor, or decrease the cross-reactivity of an inhibitor with poor specificity. By altering the R-groups in formulae VIa-d, or the number of repeating units in formula XIII, for example a skilled person can tune the affinity and specificity of the present compounds as inhibitors of cell proliferation.

A proliferative disorder relates to an unwanted or uncontrolled cellular proliferation of excessive or abnormal cells which is undesired, such as, neoplastic or hyperplastic growth, whether in vitro or in vivo. Examples of proliferative conditions include, but are not limited to, pre-malignant and malignant cellular proliferation, including but not limited to, malignant neoplasms and tumours, cancers, leukemias, psoriasis, bone diseases, fibroproliferative disorders (e.g., of connective tissues), and atherosclerosis. Any type of cell may be treated, including but not limited to, lung, colon, breast, ovarian, prostate, liver, pancreas, brain, and skin. Antiproliferative compounds of the present invention may have application in the treatment of cancer, and so the present invention further provides anticancer agents. The anti-cancer effect may arise through one or more mechanisms, including but not limited to, the regulation of cell proliferation, the inhibition of angiogenesis (the formation of new blood vessels), the inhibition of metastasis (the spread of a tumour from its origin), the inhibition of invasion (the spread of tumour cells into neighbouring normal structures), or the promotion of apoptosis (programmed cell death).

The compounds of the present invention may also be used in the treatment of conditions such as, but not limited to, cancer, psoriasis, fibroproliferative disorders (e.g., liver fibrosis), smooth muscle proliferative disorder (e.g., atherosclerosis, restenosis), neurodegenative diseases (e.g., Alzheimer's disease, Parkinson's disease, Huntington's chorea, amyotropic lateral sclerosis, spino-cerebellar degeneration), inflammatory disease (e.g., osteoarthritis, rheumatoid arthritis) diseases involving angiogenesis (e.g., cancer, rheumatoid arthritis, psoriasis, diabetic retinopathy), haematopoietic disorders (e.g., anaemia, sickle cell anaemia, thalassaeimia) fungal infection, parasitic infection (e.g., malaria, trypanosomiasis, helminthiasis, protozoal infections, bacterial infection, viral infection, conditions treatable by immune modulation (e.g., multiple sclerosis, autoimmune diabetes, lupus, atopic dermatitis, allergies, asthma, allergic rhinitis, inflammatory bowel disease; and for improving grafting of transplants).

In therapeutic use, the active compound may be administered orally, intravenously, rectally, parenterally, by inhalation (pulmonary delivery), topically, ocularly, nasally, or to the buccal cavity. Thus, a composition of the present invention may take the form of any of the known pharmaceutical compositions for such methods of administration. The compositions may be formulated in a manner known to those skilled in the art so as to give a controlled release, for example rapid release or sustained release, of the compounds of the present invention. Pharmaceutically acceptable carriers suitable for use in such compositions are well known in the art. The compositions of the invention may contain 0.1-99% by weight of active compound. The compositions of the invention are generally prepared in unit dosage form. Preferably, a unit dose comprises the active ingredient in an amount of 1-500 mg. The excipients used in the preparation of these compositions are the excipients known in the art.

Appropriate dosage levels may be determined by any suitable method known to one skilled in the art. It will be understood, however, that the specific dose level for any particular patient will depend upon a variety of factors, including the activity of the specific compound employed, the age, body weight, general health and sex of the patient; diet, time of administration, route of administration, rate of excretion, drug combination and the severity of the disease undergoing treatment.

Compositions for oral administration include known pharmaceutical forms for such administration, for example tablets, troches, lozenges, aqueous or oily suspensions, dispersible powders or granules, emulsions, hard or soft capsules, or syrups and elixirs. Compositions intended for oral use may be prepared according to any method known to the art. The compositions may contain one or more agents such as sweetening agents, flavouring agents, colouring agents and preserving agents, in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example corn starch or alginic acid; binding agents, for example starch gelatin, acacia, microcrystalline cellulose or polyvinyl pyrrolidone; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredient is mixed with an inert solid diluent, for example calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinyl pyrrolidone, gum tragacanth and gum acacia; dispersing or wetting agents may be a naturally occurring phosphatide, for example lecithin, or condensation products of an alkylene oxide with fatty acids, for example polyoxyethylene stearate, or condensation products of ethylene oxide with long-chain aliphatic alcohols, for example heptadecaethyleneoxycetanol, or condensation products of ethylene oxide with partial esters derived from fatty acids, for example polyoxyethylene sorbitan monooleate. The aqueous suspensions may also contain one or more preservatives, for example ethyl or n-propyl p-hydroxybenzoate, one or more colouring agents, one or more flavouring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents, such as those set forth above, and flavouring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives. Suitable sweetening, flavouring and colouring agents may also be present.

The pharmaceutical compositions of the invention may also be in the form of oil-in-water emulsions. The oily phase may be a vegetable oil, for example olive oil or arachis oil, or a mineral oil, for example liquid paraffin, or mixtures of these. Suitable emulsifying agents may be naturally occurring gums, for example gum acacia or gum tragacanth, naturally occurring phosphatides, for example soya bean, lecithin, and esters or partial esters derived from fatty acids and hexitol anhydrides, for example sorbitan monooleate and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavouring and colouring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be in a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid, find use in the preparation of injectables.

The compounds of the invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

Compositions for topical administration may also be suitable for use in the present invention. The active compound may be dispersed in a pharmaceutically acceptable cream, ointment or gel. A suitable cream may be prepared by incorporating the active compound in a topical vehicle such as light liquid paraffin, dispersed in a aqueous medium using surfactants. An ointment may be prepared by mixing the active compound with a topical vehicle such as a mineral oil or wax. A gel may be prepared by mixing the active compound with a topical vehicle comprising a gelling agent Topically administrable compositions may also comprise a matrix in which the pharmaceutically active compounds of the present invention are dispersed so that the compounds are held in contact with the skin in order to administer the compounds transdermally.

One aspect of the invention pertains to a kit comprising (a) the active ingredient, preferably provided in a suitable container and/or with suitable packaging; and (b) instructions for use, for example, written instructions on how to administer the active compound. The written instructions may also include a list of indications for which the active ingredient is a suitable treatment.

The following Examples illustrate the invention.

### General Experimental Procedure

A solution of 8-bromoguanosine (520 mg, 1.5 mmol, 1 eqv.) in dry DMF (12 mL) was added to the substituted phenylacetylene (1.8 mmol, 1.2 eqv.) and dry triethylamine (0.63 mL, 4.5 mmol, 3 eqv.) in a vacuum dried Schlenk tube. PdCl₂ (PPh₃)₂ (11 mg, 0.015 mmol, 1 mol %) and CuI (6 mg, 0.030 mmol, 2 mol %) were added and the reaction mixture was left to stir at 110° C. for 18 h, after which time it was allowed to cool to 40° C. and the DMF removed in vacuo to leave a brown solid. The solid was transferred to a sintered glass filter and was washed with boiling water (5×200 mL), EtOAc (5×25 mL) and diethylether (2×25 mL) yielding the product as a light cream solid (0.53 g, 91%).

Representative data for 8-(2′-phenylethynyl)guanosine: Mp 234-236° C. (decomp.); νₘₐₓ (DMSO solution)/cm⁻¹ 1695 (CO), 3126 and 3328 (NH), 3421 (OH); δ_{H} (400 MHz; DMSO-d₆) 3.65 (1H, m), 3.88 (1H, m), 3.84 (1H, s), 4.19 (1H, s), 4.99 (2H, m), 5.15 (1H, br s), 5.50 (1H, d, J 6.4), 5.89 (1H, d, J 6.4), 6.62 (2H, s), 7.51 (3H, m), 7.64 (2H, m), 10.90 (1H, s); δ_{C} (128 MHz; DMSO-d₆) 61.9, 70.4, 70.8, 79.4, 85.6, 88.2, 92.6, 117.5, 120.4, 128.9, 129.3, 129.9, 131.5, 151.1, 153.9, 156.0; m/z (FAB) MH⁺ 384 (2.5%), 369, 354, 277, 185 (100%); HRMS (MH⁺): 384.1299 (Calcd. for C₁₈H₁₈N₅O₅ 384.1307).

All the modified purine derivatives have been structurally characterised in full using a combination of spectroscopic and spectrometric techniques (infra-red, nuclear magnetic resonance, mass spectrometry (low resolution and high resolution), ultra-violet spectroscopy, melting point determination and elemental analysis).

### Example 1

### Determining Optimum Synthesis Conditions

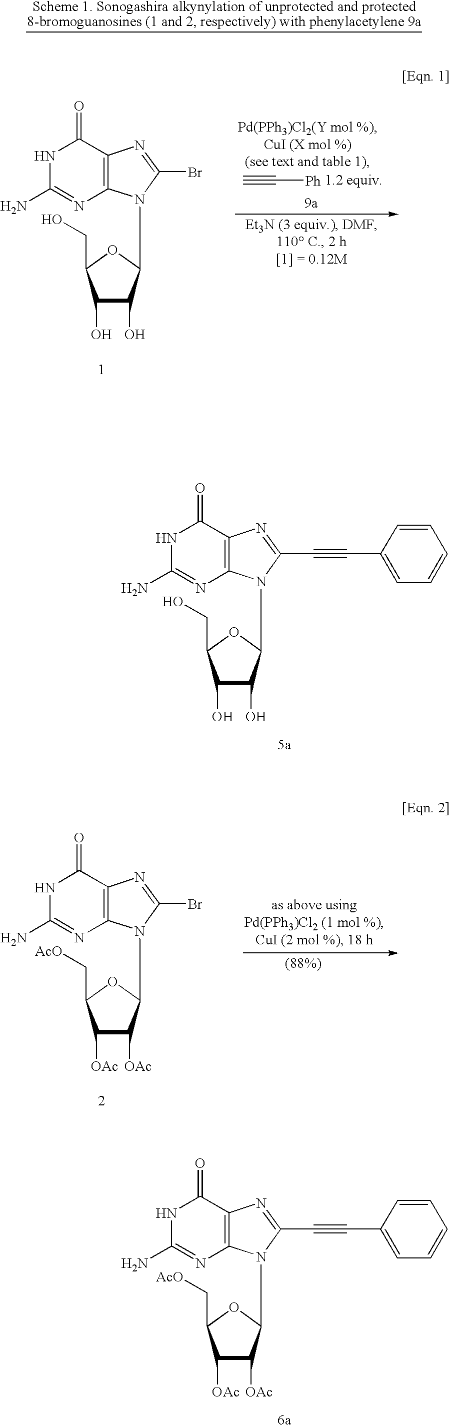

Sonogashira alkynylation protocol for the cross-coupling of 1 with phenylacetylene 9a to give 5a (Equation 1, Scheme 1). Initially, standard conditions for Sonogashira alkynylation of 1 were selected from the literature (10 mol % (Ph₃P)₂PdCl₂, 10 mol % CuI, 1.2 equiv. 9a and 3 equiv. Et₃ N in DMF at 110° C. for 18 hours). This proved to be a generally ineffective protocol, affording the product in yields of ca. 20% with poor purity (believed to be due to the uptake of Pd and/or Cu; the uptake was confirmed by X-ray photoelectron spectroscopy (XPS)), which led to optimisation of the conditions. Rapid Pd agglomeration and precipitation were thought to contribute to the low yields, which relates the optimal palladium concentration (vide supra).

Lowering the amount of (Ph₃P)₂ PdCl₂ to 5 mol % and CuI to 10% resulted in an improved yield (52%). It was established that 1 mol % (Ph₃P)₂ PdCl₂ and 2 mol % CuI proved the most effective catalyst/co-catalyst combination, affording the cross-coupled product in 95% yield (after only 2 hours). Such a successful reaction means that this reaction is suitable for scale up.

Under similar conditions, the acetate protected guanosine derivative 2 reacted with 9a to produce 6a in 88% yield, which was more cumbersome to purify than 5a, as column chromatography on silica-gel was required (eluting with methanol/chloroform solvent mixtures (1:99, v/v).

### Example 2

### Assessment of the Effect of Cu(I) on Yields

The purity of 5a was also questionable at the higher Cu(I) loading; the solid is a light green colour, rather than an off-white, due to the presence of trace quantities guanosine derived copper complexes (bidentate coordination through N-1 and O-6). This has been confirmed by infra-red spectroscopic experiments, which show the expected carbonyl shifts (in frequency) from uncoordinated ligand to coordinated ligand.

TABLE 1
Effect of Cu(I) loading on Sonogashira alkynylation of 1.

| Entry | CuI loading/mol % | Cu(I):Pd(II) ratio | Yield/% |
|---|---|---|---|
| 1 | 0 | — | 18 |
| 2 | 0.5 | 1:2 | 30 |
| 3 | 1.0 | 1:1 | 38 |
| 4 | 2.0 | 2:1 | 95 |
| 5 | 4.0 | 4:1 | 77 |

The increased catalytic activity can be explained to a certain extent by the production of higher concentrations of alkynylcuprate, although an alternative role could also be responsible for the rate enhancement vide infra.

A further experiment was carried out, where it was determined that 1 mol % PdCl₂ and 2 mol % CuI was an ineffective catalyst/co-catalyst combination, highlighting the importance of the activating phosphine ligand in the intimate steps of the catalytic cycle—it was noted that palladium nanoparticles were rapidly formed in this reaction.

### Example 3

### Purification of Product

A method for the purification of the modified purine derivatives has been developed. On complete reaction the mixture is cooled to 40° C. and the DMF removed in vacuo (at 1 mmHg) to leave a dark coloured solid, which is transferred to a sintered glass funnel and washed with boiling water (this removes Et₃ N.HCl and any unreacted starting materials from the reaction). The solid residue is then washed with small quantities of EtOAc and Et₂O to remove any traces of “organic” homocoupled product (produced through the pre-reduction of (Ph₃P)₂ PdCl₂ with alkynylcuprate). The limited solubility of the modified purine derivatives in boiling water, and the majority of chlorinated and ethereal solvents, facilitates its purification (the majority of the products are soluble in DMF, DMSO and DMSO/H₂O mixtures etc. and sonication aids their solubilisation).

### Example 4

### Alternative Synthesis

Employing 10 mol % of (Ph₃P)₂ PdCl₂ instead of 10 mol % (Ph₃P)₄ Pd, 8-bromoguanosine underwent efficient coupling with phenylacetylene to give the alkynylated product in 83% yield. The advantages associated with this technology are that the Amberlite IRA-67 can be removed at the end of the reaction by simple filtration, facilitating removal of Pd and Cu residues (Note: Amberlite IRA-67 contains basic sites as part of the resin, and does not require the addition of Et₃ N). The disadvantages are that high palladium and copper loadings are required for high yields (10 mol % and 20 mol %, respectively), and the reaction does not work so well at lower catalyst loadings. The physical form of the resin post-reaction is unique, appearing as black pellets. The surface composition of the pre-reaction and post-reaction Amberlite IRA-67 was confirmed by X-ray photoelectron spectroscopy (XPS).

The presence of free base sites within the resin, and of high concentrations of Pd(0) and Cu(I), suggests that recycling ought to be possible (without basification of the resin). In a first recycling experiment, the post-Amberlite black solid was added to a DMF solution containing 1 and 9a and heated to 110° C. for 18 h. However, this gave 5a in very poor yield (7%), an outcome suggesting that the remaining nitrogen sites in the Amberlite IRA-67 resin are not accessible (i.e. not on the surface, which has been characterised by X-ray photoelectron spectroscopy).

### Example 5

### Synthesis of Alkynated Guanosines

Using optimised conditions (1 mol % Pd(II), 2 mol % Cu(I), Et₃ N (3 equiv.), DMF, 110° C.) as described in examples 1, 2 and 3, a series of Sonogashira alkynylation reactions were performed on 8-bromoguanosine with a library of terminal acetylenes.

### Conditions

1 equiv of 8-bromoguanosine, (Ph₃P)₂ PdCl₂ (1 mol %), CuI (2 mol %), terminal acetylene (1.2 equiv.), Et₃ N (3 equiv.), DMF, 110° C., 18 h.

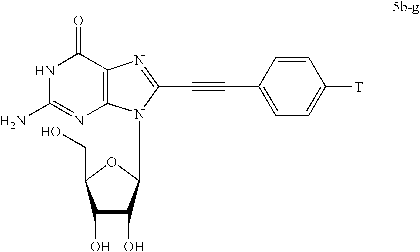

### Observations

Good yields were recorded for electron-rich phenylacetylenes 9b,c and e (entries 1, 2 and 4, Table 3). 4-Thiomethylphenylacetylene 9d gave 5d in a good 54% yield (entry 3, Table 3). The electron-deficient terminal acetylenes 9f and 9g both reacted with 8-bromoguanosine to give the products 5f and 5g in 68% and 53%, respectively (entries 5 and 6). Both 1-ethynylnaphthalene 9h and 1-ethynylferrocene 91 gave 5h and 51 in 66% and 69% yields, respectively (entries 7 and 8, Table 3).

TABLE 3
Sonogashira alkynylation of 8-bromoguanosine.

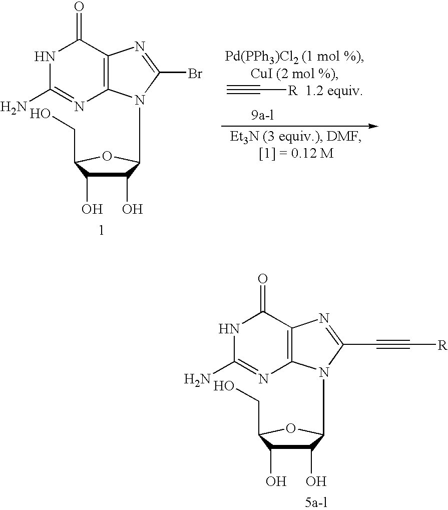

| Compound | Terminal acetylene | Product (R=)/yield (%) |
|---|---|---|
| 1 | 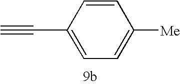 | 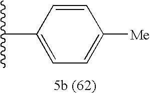 |
| 2 | 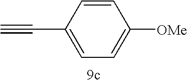 | 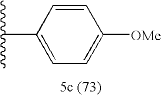 |
| 3 | 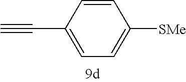 | 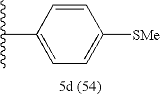 |
| 4 | 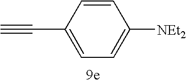 | 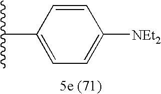 |
| 5 | 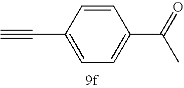 | 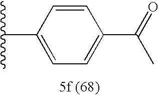 |
| 6 | 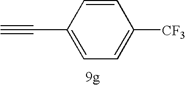 | 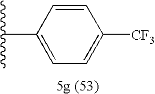 |
| 7 | 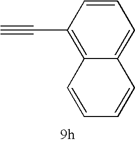 | 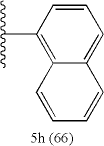 |
| 8 | 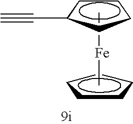 | 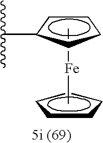 |

### Data

### 8-Bromoguanosine

(8.86 g, 70%) as a white solid: mp 220-222° C. (dec); νₘₐₓ (DMSO solution)/cm⁻¹ 1696 (CO), 3309 and 3421 (NH), 3505 (OH); m/z (CI) M-H₂O 344 (0.2%), 230 (100%), 150, 132, 115.

### 8-Bromoguanosine-2′,3′,5′-triacetate

(2.15 g, 32%) as a white solid: νₘₐₓ (DMSO solution)/cm⁻¹ 1697 (CO), 1747 (CO) 3164 and 3326 (NH); m/z (CI) M 488 (1.0%), 259, 217, 199, 156, 139 (100%).

### 8-(Phenylethynyl)guanosine-2′,3′,5′-triacetate

(0.67 g, 88%) as a yellow solid: νₘₐₓ (DMSO solution)/cm⁻¹ 1697 (CO), 1747 (CO) 3166 and 3316 (NH); m/z (FAB) MH⁺ 510 (27%), 252, 155 (100%), 136

### 8-(Phenylethynyl)guanosine

(0.53 g, 91%) as a yellow solid; mp 234-236° C. (dec); IR (DMSO Solution): ν^{˜}=3421 cm⁻¹ (OH), 3328 cm⁻¹ (NH), 3126 cm⁻¹ (NH), 1695 cm⁻¹ (CO); MS (FAB) m/z (%): 384 (2.5) [MH⁺], 369, 354, 277, 185(100). HR (MH⁺): 384.1299 (Calculated for C₁₈H₁₈N₅O₅ 384.1307).

### 8-(4′-Methylphenylethynyl)guanosine

(0.37 g, 62%) as a brown/yellow solid; mp 222-226° C. (dec); ν^{˜}=3521 cm⁻¹ (OH), 3434 cm⁻¹ (NH), 3301 cm⁻¹ (NH), 1691 cm⁻¹ (OH); MS (FAB) m/z (%): 398 (20.0) [MH⁺], 382, 277, 260, 250, 185(100). HR (MH⁺): 398.1466 (Calculated for C₁₉H₂₀N₅O₅ 398.1464).

### 8-(4′-Acetophenonephenylethynyl)guanosine

(0.43 g, 68%) as a yellow solid; mp 224-228° C. (dec); ν^{˜}=3430 cm⁻¹ (OH), 3299 cm⁻¹ (NH), 3152 cm⁻¹ (NH), 1683 cm⁻¹ (CO); MS (FAB) m/z (%): 426 (3.0) [MH⁺], 369, 277, 185(100). HR (MH⁺): 426.1408 (Calculated for C₁₉H₂₀N₅O₅ 426.1414).

### 8-(Napthylethynyl)guanosine

(0.51 g, 66%) as a brown solid; mp 234-238° C. (dec); IR (DMSO Solution): ν^{˜}=3441 cm⁻¹ (OH), 3316 cm⁻¹ (NH), 3120 cm⁻¹ (NH), 1696 cm⁻¹ (CO); MS (FAB) m/z (%): 434 (8.0) [MH⁺], 400, 364, 302 (100); HR (MH⁺): 434.1456 (Calculated for C₂₂H₂₀N₅O₅ 434.1464).

### 8-(4′-Trifluoromethylphenylethynyl)guanosine

(0.36 g, 53%) as a creme solid; mp>240° C. IR (DMSO Solution): ν^{˜}=3489 cm⁻¹ (OH), 3310 cm⁻¹ (NH), 3131 cm⁻¹ (NH), 1696 cm⁻¹ (CO), 1324 cm⁻¹ (CF); MS (FAB) m/z (%): 452 (2.0) [MH⁺], 369, 277, 185 (100); HR (MH⁺): 452.1176 (Calculated for C₁₉H₁₇N₅O₅F₃ 452.1182).

### 8-(4-N,N-diethylaminephenylethynyl)guanosine

(0.49 g, 71%) as a brown/yellow solid: mp 190-196° C. (dec); IR (DMSO Solution): ν^{˜}=3443 cm⁻¹ (OH), 3313 cm⁻¹ (NH), 3214 cm⁻¹ (NH), 1691 cm⁻¹ (CO); MS (FAB) m/z (%): 455 (23) [MH⁺], 323, 277, 185(100). HR (MH⁺): 455.2046 (Calculated for C₂₂H₂₇N₆O₅ 455.2043).

### 8-(4-Methoxyphenylethynyl)guanosine

(0.45 g, 73%) as a creme solid: mp 220-224° C. (dec); IR (DMSO Solution): ν^{˜}=3428 cm⁻¹ (OH), 3304 cm⁻¹ (NH), 3230 cm⁻¹ (NH), 1696 cm⁻¹ (CO); MS (FAB) m/z (%): 414 (4.0) [MH⁺], 369, 277, 185(100). HR (MH⁺): 414.1413 (Calculated for C₁₉H₂₀N₅O₆ 414.1414).

### 8-(4-Methylsulfanylphenylethynyl)guanosine

(0.34 g, 54%) as a grey solid: mp 214-216° C. (dec); IR (DMSO Solution): ν^{˜}=3430 cm⁻¹ (OH), 3309 cm⁻¹ (NH), 3201 cm⁻¹ (NH), 1696 cm⁻¹ (CO); MS (FAB) m/z (%): 430 (24) [MH⁺], 298, 277, 185(100). HR (MH⁺): 430.1179 (Calculated for C₁₉H₂₀N₅O₅S 430.1185).

### 8-(1′-Ethynylferrocene)guanosine

(0.45 g, 69%) as a brown solid: mp 224-228° C. (dec); νₘₐₓ (DMSO solution)/cm⁻¹ 1690 (CO), 3170 and 3299 (NH), 3430 (OH); m/z (FAB) MH⁺⁴⁹² (18%), 360 (100%), 296.

### Optical Properties

| Compound | λₑₓ/nm | λₑₘ/nm | Stokes shift^{a}/cm⁻¹ |
|---|---|---|---|
| 5a | 370 | 438 | 4196 |
| 5b | 363 | 415 | 3452 |
| 5c | 362 | 402 | 2749 |
| 5d | 374 | 426 | 3264 |
| 5e | 398 | 502 | 5205 |
| 5f | 397 | 532 | 6392 |
| 5g | 386 | 468 | 4539 |
| 5h | 383 | 465 | 4604 |
| 5i | 380 | 439 | 3537 |

### Example 6

### Synthesis of Alkynated Adenosines

Using these optimised conditions as described in Examples 1, 2 and 3, a series of Sonogashira alkynylation reactions were performed on 8-bromoadenosine.

### Conditions

1 equiv. 8-bromoadenosine), (Ph₃P)₂ PdCl₂ (1 mol %), CuI (2 mol %), terminal acetylene (1.2 equiv.), Et₃ N (3 equiv.), DMF, 110° C., 18 h.

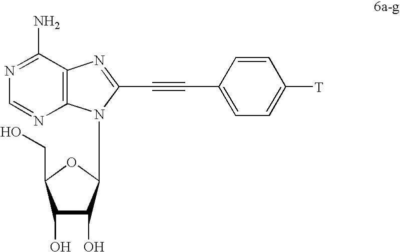

### Observations

Good to high yields were recorded for compounds 6a-d (entries 1-4, Table 4). The 4-diethylaminophenyl derivative 6e was produced in a modest 52% yield (entry 5, Table 4). The 4-acetophenone derivative 6f and 4-trifluoromethylphenyl derivative 6g were prepared in 74% and 64% yield, respectively (entries 6 and 7, Table 4). 1-Ethynylnaphthalene 9h coupled satisfactorily to give 6h in 57% yield (entry 8, Table 4), as did 1-ethynylferrocene 9i to give 6i in 58% yield (entry 9, Table 4).

TABLE 4
Sonogashira alkynylation of 8-bromoadenosine.

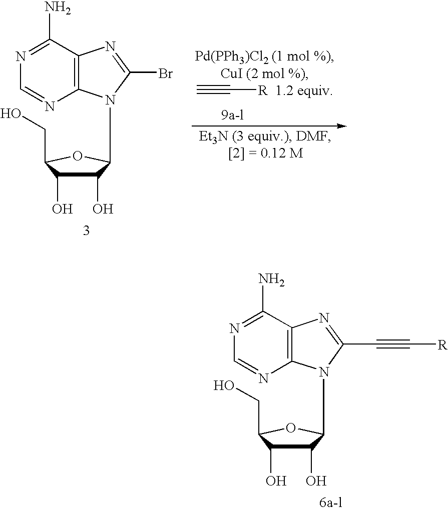

| Compound | Terminal acetylene | Product (R=)/yield (%) |
|---|---|---|
| 1 | 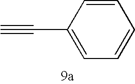 | 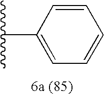 |
| 2 | 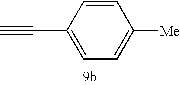 | 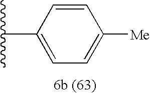 |
| 3 | 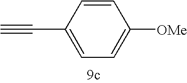 | 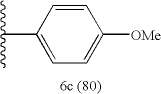 |
| 4 | 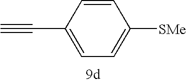 | 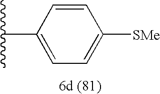 |
| 5 | 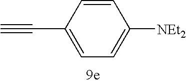 | 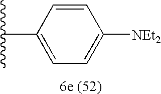 |
| 6 | 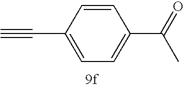 | 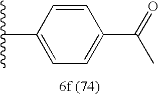 |
| 7 | 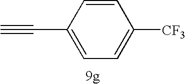 | 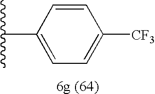 |
| 8 | 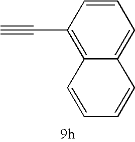 | 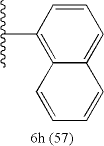 |
| 9 | 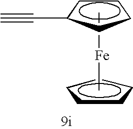 | 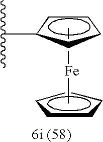 |

### Data

### 8-Bromoadenosine

(2.75 g, 39%) as a pale yellow solid: mp 210-212° C. (dec); νₘₐₓ (DMSO solution)/cm⁻¹ 3089 br (NH), 3327 br (OH); m/z (CI) M 346 (21%), 348, 214 (100%), 150, 136.

### 8-(Phenylethynyl)adenosine

(0.47 g, 85%) as a yellow solid; mp 168-170° C. (dec); IR (DMSO Solution): ν^{˜}=3431 cm⁻¹ (OH), 3320 cm⁻¹ (NH), 3183 cm⁻¹ (NH); MS (FAB) m/z (%): 368 (4.5) [MH⁺], 277, 185(100). HR (MH⁺): 368.1366 (Calculated for C₁₈H₁₈N₅O₄ 368.1359).

### 8-(4′-Methylphenylethynyl)adenosine

(0.36 g, 63%) as a brown/yellow solid; mp 196-198° C. (dec); IR (DMSO Solution): ν^{˜}=3407 cm⁻¹ (OH), 3310 cm⁻¹ (NH), 3195 cm⁻¹ (NH); MS (FAB) m/z (%): 382 (59.0) [MH⁺], 277, 250, 185(100). HR (MH⁺): 382.1514 (Calculated for C₁₉H₂₀N₅O₄ 382.1515).

### 8-(4′-Acetophenonephenylethynyl)adenosine

(0.45 g, 57%) as a brown solid; mp 208-210° C. (dec); IR (DMSO Solution): ν^{˜}=3456 cm⁻¹ (OH), 3286 br cm⁻¹ (NH), 1681 cm⁻¹ (CO); MS (FAB) m/z (%): 410 (9.0) [MH⁺], 369, 277, 185(100). HR (MH⁺): 410.1467 (Calculated for C₂₀H₂₀N₅O₄ 410.1462).

### 8-(Napthylethynyl)adenosine

(0.42 g, 57%) as a beige solid; mp 190-196° C. (dec); IR (DMSO Solution): ν^{˜}=3456 cm⁻¹ (OH), 3304 cm⁻¹ (NH), 3175 cm⁻¹ (NH); MS (FAB) m/z (%): 418 (50.0) [MH⁺], 286, 277, 185(100). HR (MH⁺): 418.1510 (Calculated for C₂₂H₂₀N₅O₄ 418.1515).

### 8-(4′-Trifluoromethylphenylethynyl)adenosine

(0.42 g, 64%) as a brown/orange solid; mp 196-198° C. (dec); IR (DMSO Solution): ν^{˜}=3442 cm⁻¹ (OH), 3311 cm⁻¹ (NH), 3150 cm⁻¹ (NH), 1324 cm⁻¹ (CF); MS (FAB) m/z (%): 436 (15.0) [MH⁺], 369, 304, 277, 185(100). HR (MH⁺): 436.1239 (Calculated for C₁₉H₁₇N₅O₄F₃ 436.1233).

### 8-(4-N,N-Diethylaminephenylethynyl)adenosine

(0.34 g, 52%) as a brown solid: mp 210-214° C. (dec); IR (DMSO Solution): ν^{˜}=3432 cm⁻¹ (OH), 3330 cm⁻¹ (NH), 3165 cm⁻¹ (NH); MS (FAB) m/z (%): 439 (40) [MH⁺], 307, 277, 185(100). HR (MH⁺): 439.2099 (Calculated for C₂₂H₂₇N₆O₄ 439.2094).

### 8-(4-methoxyphenylethynyl)-adenosine

(0.44 g, 80%) as a creme/yellow solid: mp 198-202° C. (dec); IR (DMSO Solution): ν^{˜}=3409 cm⁻¹ (OH), 3302 cm⁻¹ (NH), 3178 cm⁻¹ (NH); MS (FAB) m/z (%): 398 (52) [MH⁺], 277, 266, 185(100). HR (MH⁺): 398.1463 (Calculated for C₁₉H₂₀N₅O₅ 398.1464).

### 8-(4-Methylsulfanylphenylethynyl)adenosine

(0.50 g, 81%) as a brown solid: mp 184-188° C. (dec); IR (DMSO Solution): ν^{˜}=3427 cm⁻¹ (OH), 3304 cm⁻¹ (NH), 3178 cm⁻¹ (NH); MS (FAB) m/z (%): 414 (41) [MH⁺], 385, 282 (100). HR (MH⁺): 414.1236 (Calculated for C₁₉H₂₀N₅O₄S 414.1236).

### 8-(1′-Ethynylferrocene)adenosine

(g, %) as a brown solid: mp ° C. (dec); νₘₐₓ (DMSO solution)/cm⁻¹ 3231 and 3365 (NH), 3457 br (OH); δ_{H} (400 MHz; DMSO-d₆); δ_{C} (128 MHz; DMSO-d₆); m/z (FAB) MH⁺476 (17.0%), 369, 344, 277, 185 (100%); HR (MH⁺):

### Optical Properties

| Compound | λₑₓ/nm | λₑₘ/nm | Stokes shift^{a}/cm⁻¹ |
|---|---|---|---|
| 6a | 350 | 397 | 3383 |
| 6b | 350 | 393 | 3126 |
| 6c | 354 | 380 | 1933 |
| 6d | 368 | 396 | 1921 |
| 6e | 408 | 506 | 4747 |
| 6f | 374 | 493 | 6454 |
| 6g | 362 | 440 | 4897 |
| 6h | 376 | 431 | 3394 |
| 6i | 370 | 434 | 3986 |

### Example 7

### Optical Properties

### Experimental

DMF was dried over molecular sieves, distilled and stored under N₂ in a large ampule. All reactions were conducted under an inert atmosphere of Ar or N₂ on a Schlenk line (with high vacuum capacity). Melting points were recorded on an electrothermal IA9000 Digital Melting Point Apparatus and are uncorrected. TLC analysis was performed on Merck 5554 aluminium backed silica gel plates and compounds visualized by ultraviolet light (254 nm), phosphomolybdic acid solution (5% in EtOH), or 1% ninhydrin in EtOH. ¹H and ¹³C spectra were recorded on a Jeol EX270 spectrometer operating at 270 and 67.9 MHz respectively, on a Bruker 500 at 500 and 125.7 MHz, respectively, or a Jeol ECX400 spectrometer operating at 400 and 100.6 MHz, respectively. ³¹P NMR were recorded on a Jeol EX270, Jeol ECX400 or Bruker 500 spectrometers operating at 109.1, 161.2, 202.1 MHz, respectively. The solutions were prepared in suitable deuterated solvents. Referencing of samples was accomplished by using either TMS or residual protonated solvent. Chemical shifts are reported in parts per million downfield from an internal tetramethylsilane reference. Coupling constants (J values) are reported in hertz (Hz), and spin multiplicities are indicated by the following symbols: s (singlet), d (doublet), t (triplet), q (quartet), qn (quintet), m (multiplet), br (broad). All melting points were recorded on an Electrotherminal IA9000 digital melting point apparatus and were uncorrected. Low- and high-field resolution electron ionisation (EI), chemical ionisation (CI) and fast atom bombardment (FAB) mass spectrometry were performed using a Fisons analytical (VG) Autospec instrument. All of the values are reported in accordance with convention and high resolution molecular ions given are within ±5 ppm of the required molecular mass. Infra-red spectral data were obtained using an ATI Mattson Genesis FT-IR spectrometer a Thermo Nicolet IR 100.

The optical properties of several novel chromophores were investigated. UV-visible spectra reveal an intense absorption in the UV region for all the compounds evaluated. As expected, the spectrum of chromophore 5c is red-shifted from that of compound 5a (the parent compound), revealing a smaller HOMO-LUMO gap. Compound 5e, also red-shifted relative to 5a, exhibits a higher HOMO-LUMO gap than 5a. Similar trends are seen with the adenosine compounds 7a, 7c and 7e, although more significant differences are observed in the absorption maxima shift of 7c and 7e relative to compound 7a (the parent compound).

| Compound | λₑₓ/nm | λₑₘ/nm | Stokes shift^{a}/cm⁻¹ |
|---|---|---|---|
| 5a | 370 | 438 | 4196 |
| 5b | 363 | 415 | 3452 |
| 5c | 362 | 402 | 2749 |
| 5d | 374 | 426 | 3264 |
| 5e | 398 | 502 | 5205 |
| 5f | 397 | 532 | 6392 |
| 5g | 386 | 468 | 4539 |
| 5h | 383 | 465 | 4604 |
| 5i | 380 | 439 | 3537 |
| 6a | 350 | 397 | 3383 |
| 6b | 350 | 393 | 3126 |
| 6c | 354 | 380 | 1933 |
| 6d | 368 | 396 | 1921 |
| 6e | 408 | 506 | 4747 |
| 6f | 374 | 493 | 6454 |
| 6g | 362 | 440 | 4897 |
| 6h | 376 | 431 | 3394 |
| 6i | 370 | 434 | 3986 |

^{a}Stokes shift = (1/λₑₓ − 1 λₑₘ).

### Example 8

In vitro assays of hepatitis C virus (HCV) and bacteriophage T7 RNA polymerases have been performed using adenosine ribonucleotide triphosphate analogues of the present invention as a substrate (in place of natural ATP). Both unincorporated and incorporated fluorescent purine ribonucleotide analogues could be visualised directly under UV trans-illumination in either agarose or polyacrylamide gels without ethidium bromide staining. The RNA products generated in the transcription assay were characterised by 6% or 20% polyacrylamide gel electrophoresis (PAGE) under denaturing (7 M urea) conditions. For example, results with HCV and T7 RNA polymerases using 8-phenylethynyl-adenosine triphosphate as a substrate (and Mg²⁺ as a co-factor) yielded short RNA transcripts depending on the DNA template employed (see the Table below). The expected full-length (˜500 bases) RNA products were not observed by denaturing 6% PAGE. Short RNA products were separated by denaturing 20% PAGE. These gels demonstrated that the analogue is incorporated into small RNA products (4-9 bases in length), probably during abortive cycling by the RNA polymerase. The analogues are also incorporated at a slower rate than the natural substrates. Incorporation of the first and second adenosine (underlined adenosine residues in the Table below) into the RNA product occurs with two templates tested, but adjacent adenosine analogues terminate transcription before the third adenosine in each template is incorporated. Two adjacent adenosine analogues may be too large for the active site, thus RNA synthesis is terminated and the product released.

RNA products formed using the HCV 3′-UTR and T7 φ13 promoter DNA templates in gel-based transcription assays of T7 RNA polymerase and 8-phenylethynyl-adenosine triphosphate:

| | | Length of |
|---|---|---|
| DNA template type | Initial transcribed region* | RNA products observed |
| HCV 3′-UTR | GGGCCACCAACGAUCUGACCGCC . . . | 6 mer, 9 mer |
| T7 φ13 promoter | GGGAGAACAATACGACTACGGGA . . . | 4 mer, 6 mer |

*The underlined bases denote the two positions where the adenosine triphosphate analogue is incorporated based on polyacrylamide gel electrophoresis assays.

The level of incorporation may be improved by using a mixture of natural ATP and fluorescent ATP analogue, and/or by using Mn²⁺ as a co-factor. Mn²⁺ has a smaller atomic radius and often supports transcription with base-modified ribonucleotide analogues. Its smaller atomic radius may reduce steric effects in the active site induced by two adjacent analogues in the RNA transcript. These modifications to the transcription assay conditions could also be used in conjunction with other polymerases and/or other classes of fluorescent analogues in the present invention.

### Example 9

UV absorbance and fluorescence excitation and emission data were used to determine the molar extinction coefficient and fluorescence quantum yield of a several adenosine and guanosine ribonucleoside analogues in DMSO (see the Table below). The data acquired support the concept that the fluorescence properties of these analogues can be tuned by altering the electron-donating or electron-withdrawing R-group on the terminal phenylalkyne. Fourteen electron-donating or electron-withdrawing R-groups were coupled to purine bases at the 8-position. All of the analogues were found to have absorption maxima that are far removed from the absorption peaks for protein (˜280 nm) and π-π* transition in nucleic acids (˜260 nm). This makes the analogues extremely useful for the quantification of analogue-containing nucleic acid products in a DNA or RNA background. The molar extinction coefficient results obtained for the para substitutents do not correlate with the Hammett values for the R-groups. This suggests that the purine base may act as either an electron-donating or electron-withdrawing moiety. The position (ortho, meta or para) of the R-group on the phenyl ring can also be used to tune the fluorescent properties of the analogues. The ring position of the R-group has a smaller effect on the fluorescent properties of the analogues. Greater changes in the excitation/emission wavelength, molar extinction coefficient and quantum yield can be induced by changing the type of R-group (electron-donating vs. electron-withdrawing) as compared to the position of a R-group on the phenyl ring (ortho, meta or para). The quantum yields of the unphosphorylated analogues were measured relative to quinine sulphate and anthracene standards. These external standards were chosen because they possess excitation and emission wavelengths in the same region of the photonic spectrum as the analogues. Results show the adenosine nucleoside analogues possess greater quantum efficiency as compared to the guanosine nucleoside analogues. As seen for the molar extinction coefficients, the quantum yields obtained for the para substitutents do not correlate with the Hammettt values for the R-groups. This provides additional support for the idea that the purine bases can both push and pull electrons in π-conjugated systems.

| | | | Wavelength at | Molar | Wavelength at | |
|---|---|---|---|---|---|---|
| | | | maximum in UV | absorption | fluorescence | |
| | | Position on | absorption | coefficient | emission maximum | Quantum yield |
| Purine base | R- group | phenyl ring | (nm) | (M⁻¹ cm⁻¹) | (nm) | (%) |
| Adenosine | —Cl | para | 326 | 23927 | 410 | 46 |
| Adenosine | —COCH₃ | para | 343 | 19639 | 493 | 4.9 |
| Adenosine | —CF₃ | para | 333 | — | 436 | 8.8 |
| Adenosine | —CF₃ | meta | 326 | 19547 | 421 | 21 |
| Adenosine | —CF₃ | ortho | — | — | — | — |
| Adenosine | —F | para | 320 | 5558 | 396 | 40 |
| Adenosine | —H | para | 322 | — | 398 | 49 |
| Adenosine | —SCH₃ | para | 338 | 26019 | — | 67 |
| Adenosine | —CH₃ | para | 323 | 18772 | 393 | 54 |
| Adenosine | —OCH₃ | para | 326 | 18176 | 380 | 24 |
| Adenosine | —OCH₃ | meta | 324 | 19589 | 400 | 52 |
| Adenosine | —OCH₃ | ortho | 333 | — | 429 | 21 |
| Adenosine | —N(CH₂CH₃)₂ | para | 365 | — | 441 | 8.4 |
| Adenosine | naphthalene | para | 349 | 22970 | 430 | 49 |
| Guanosine | —Cl | para | 338 | 23317 | 431 | 1.9 |
| Guanosine | —COCH₃ | para | 364 | 19589 | 509 | 0.44 |
| Guanosine | —CF₃ | para | 348 | — | 458 | 0.74 |
| Guanosine | —CF₃ | meta | 342 | — | 443 | 1.5 |
| Guanosine | —CF₃ | ortho | 347 | — | 421 | 1.1 |
| Guanosine | —F | para | 333 | 11304 | 416 | 2.4 |
| Guanosine | —H | para | 330 | — | 416 | 2.7 |
| Guanosine | —SCH₃ | para | 343 | 19832 | 425 | 4.5 |
| Guanosine | —CH₃ | para | 331 | 16591 | 409 | 3.1 |
| Guanosine | —OCH₃ | para | 330 | 22569 | 398 | 1.2 |
| Guanosine | —OCH₃ | meta | 334 | 20261 | 420 | 3.6 |
| Guanosine | —OCH₃ | ortho | — | — | — | — |
| Guanosine | —N(CH₂CH₃)₂ | para | 356 | — | 409 | 2.1 |
| Guanosine | naphthalene | para | 357 | 15141 | 467 | 5.4 |

1. A compound of the formula (I): 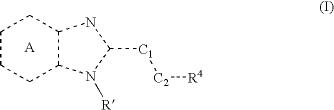
wherein
ring A selected from; 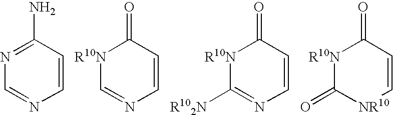
wherein the or each R¹⁰ is independently hydrogen, lower alkyl, lower acyl or a sugar residue;
the broken lines ------ represent a delocalized electron system where electronic communication is possible;
C₁---C₂ is an alkenylene or alkynylene linker;
R⁴ is selected from cyclopentadienyl, phenyl, naphthyl, pyridinyl, pyrazinyl, pyrimidinyl, triazinyl, thiophenyl, parathiazinyl, pyrrolyl, pyrazolyl, imidazolyl, indolyl, purinyl, benzimidazolyl, quinolinyl and phthalazinyl, any of which is substituted or unsubstituted; or R⁴ comprises a ring system comprising one or more metal atoms; and
R′ is a sugar residue.

2. The compound of claim 1, wherein the compound has the formula: 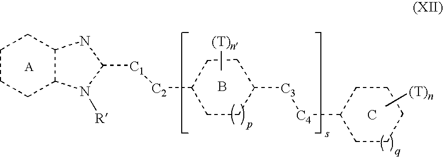
wherein
rings B and C are each independently a five- or six-membered carbocyclic or heterocyclic ring;
the or each C₃---C₄ is independently an alkenylene or alkynylene linker;
the or each T is independently selected from halogen; hydroxy; protected hydroxy for example trialkylsilylhydroxy or etherified or esterified hydroxy; amino; mono- or di-substituted amino; amidino; guanidino; hydroxyguanidino; formamidino; N-hydroxy amidino; isothioureido; ureido; mercapto; C(O)H or other lower acyl; lower acyloxy; carboxy; esterified carboxy; sulfo; sulfamoyl; carbamoyl; cyano; azo (N═N═N); nitro; O-lower alkyl; or lower alkyl; O-lower alkyl and lower alkyl optionally substituted by one or more halogens and/or one or two functional groups selected from hydroxy, protected hydroxy for example trialkylsilylhydroxy, amino, mono- or di-substituted amino, amidino, guanidino, hydroxyguanidino, formamidino, isothioureido, ureido, mercapto, C(O)H or other lower acyl, lower acyloxy, carboxy, sulfo, sulfamoyl, carbamoyl, cyano, azo, or nitro; all of which hydroxy, amino, amidino, guanidino, hydroxyguanidino, formamidino, isothioureido, ureido, mercapto, carboxy, sulfo, sulfamoyl, carbamoyl and cyano groups being optionally substituted, where appropriate;
p, q and s are each independently 0 or 1;
n is 0, 1, 2, 3, 4 or 5; and
n′ is 0, 1, 2, 3 or 4.

3. (canceled)

4. (canceled)

5. The compound of claim 2, wherein the compound has a formula selected from:

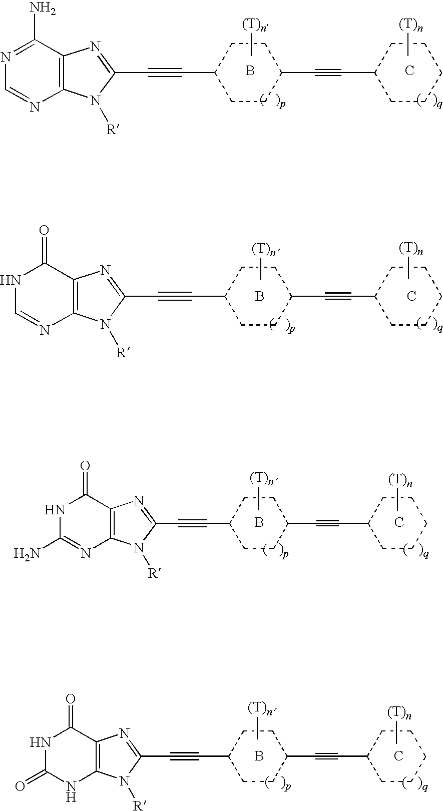

6. The compound of claim 1, wherein R′ is a sugar residue and has the formula 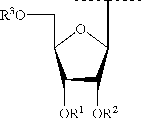
wherein R¹, R² and R³ are each independently selected from hydrogen, lower alkyl, halo-lower alkyl, carboxy, lower alkanoyl and a counter ion to the ion, O⁻ or one or more phosphate groups; or R¹ and R³ taken together may form a cyclic monophosphate derivative.

7. The compound of claim 6, where R³ comprises at least one phosphate group.

8. The compound of claim 1, wherein R⁴ is phenyl, napthyl or thiophenyl, any of which is optionally substituted by one or more substituents T; wherein the or each T is independently selected from halogen; hydroxy; protected hydroxy for example trialkylsilylhydroxy or etherified or esterified hydroxy; amino; mono- or di-substituted amino; amidino; guanidino; hydroxyguanidino; formamidino; N-hydroxy amidino; isothioureido; ureido; mercapto; C(O)H or other lower acyl; lower acyloxy; carboxy; esterified carboxy; sulfo; sulfamoyl; carbamoyl; cyano; azo (N═N═N); nitro; O-lower alkyl; or lower alkyl; O-lower alkyl and lower alkyl optionally substituted by one or more halogens and/or one or two functional groups selected from hydroxy, protected hydroxy for example trialkylsilylhydroxy, amino, mono- or di-substituted amino, amidino, guanidino, hydroxyguanidino, formamidino, isothioureido, ureido, mercapto, C(O)H or other lower acyl, lower acyloxy, carboxy, sulfo, sulfamoyl, carbamoyl, cyano, azo, or nitro; all of which hydroxy, amino, amidino, guanidino, hydroxyguanidino, formamidino, isothioureido, ureido, mercapto, carboxy, sulfo, sulfamoyl, carbamoyl and cyano groups being optionally substituted, where appropriate.

9. The compound of claim 35, wherein the compound has a formula selected from:

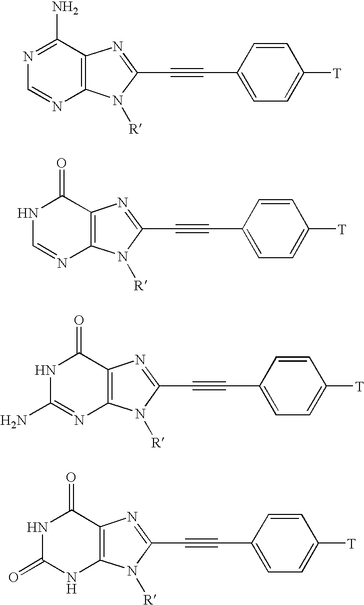

10. The compound of claim 9, wherein the compound has the formula: 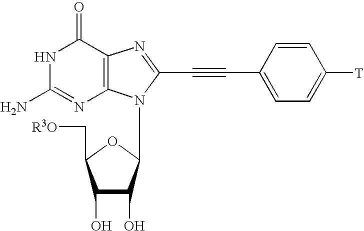
wherein 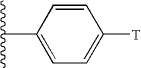
is selected from:

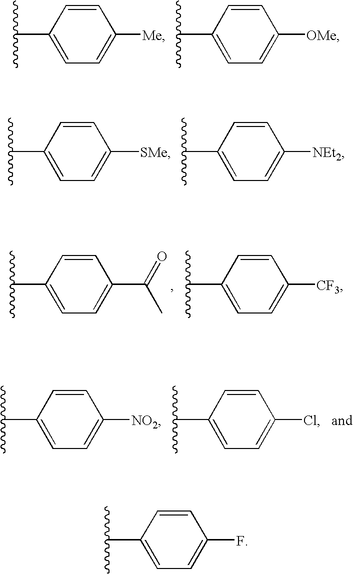

11. The compound of claim 8, wherein the compound has the formula: 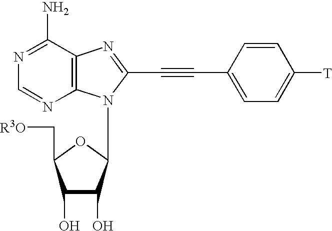
wherein 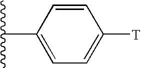
is selected from:

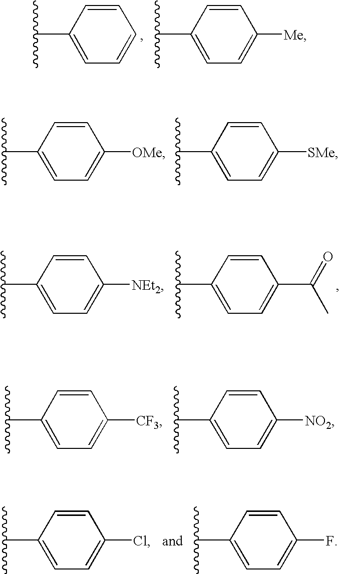

12. The compound of claim 1, wherein electronic communication is possible between ring A and R⁴.

13. The compound of claim 1, wherein said broken lines represent a conjugated π-network.

14. (canceled)

15. (canceled)

16. (canceled)

17. (canceled)

18. (canceled)

19. (canceled)

20. (canceled)

21. (canceled)

22. (canceled)

23. A method for making a compound of claim 1 comprising reacting a halogenated purine adduct with a terminal alkyne in the presence of 1 mol % Pd(II), 2 mol % Cu(I), Et₃ N (3 equiv.).

24. A method for making a compound of claim 1 comprising reacting a halogenated purine adduct with a terminal alkyne in the presence of from 1×10⁻³ to 10⁻⁴ Mol dm⁻³ Pd(II).

25. (canceled)

26. (canceled)

27. (canceled)

28. (canceled)

29. (canceled)

30. (canceled)

31. (canceled)

32. (canceled)

33. (canceled)

34. (canceled)

35. The compound of claim 1, wherein the compound has a formula selected from: 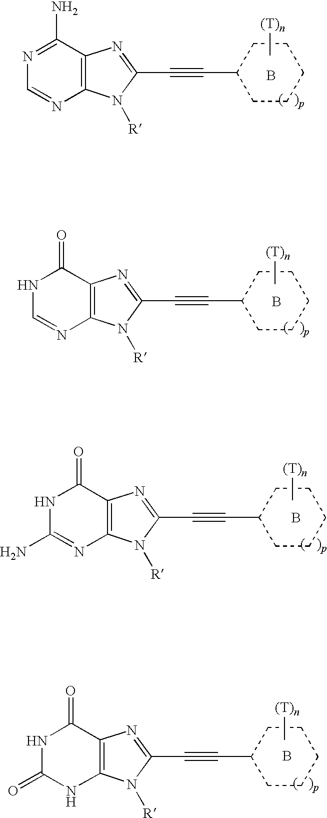
wherein
n is 0, 1, 2, 3, 4 or
p is 0 or 1;
ring B is a five- or six-membered carbocyclic or heterocyclic ring; and the or each T is independently selected from halogen; hydroxy; protected hydroxy for example trialkylsilylhydroxy or etherified or esterified hydroxy; amino; mono- or di-substituted amino; amidino; guanidino; hydroxyguanidino; formamidino; N-hydroxy amidino; isothioureido; ureido; mercapto; C(O)H or other lower acyl; lower acyloxy; carboxy; esterified carboxy; sulfo; sulfamoyl; carbamoyl; cyano; azo (N═N═N); nitro; O-lower alkyl; or lower alkyl; O-lower alkyl and lower alkyl optionally substituted by one or more halogens and/or one or two functional groups selected from hydroxy, protected hydroxy for example trialkylsilylhydroxy, amino, mono- or di-substituted amino, amidino, guanidino, hydroxyguanidino, formamidino, isothioureido, ureido, mercapto, C(O)H or other lower acyl, lower acyloxy, carboxy, sulfo, sulfamoyl, carbamoyl, cyano, azo, or nitro; all of which hydroxy, amino, amidino, guanidino, hydroxyguanidino, formamidino, isothioureido, ureido, mercapto, carboxy, sulfo, sulfamoyl, carbamoyl and cyano groups being optionally substituted, where appropriate.
